(19) United States
(12) Patent Application Publication
(10) Pub. No.: US 2026/0284207 A1
(43) Pub. Date: Sep. 24, 2026

(54) COMPOUND FOR USE AS A PEPTIDE BINDER SPECIFICALLY BINDING TO THE UBIQUITIN LIGASE ADAPTER FBXO31

(71) Applicant: ETH ZÜRICH, Zürich (CH)

(72) Inventors: Jeffrey BODE, Zürich (CH); Raphael HOFMANN, New York, NY (US); Jacob Elley CORN, Zürich (CH); Matthias MUHAR, Zürich (CH); Jakob FARNUNG, Dortmund (DE)

(73) Assignee: ETH Zürich, Zürich (CH)

(21) Appl. No.: 19/134,552

(22) PCT Filed: Dec. 1, 2023
(86) PCT No.: PCT/EP2023/083933

§ 371 Date: May 30, 2025

(30) Foreign Application Priority Data
Dec. 1, 2022 (EP) 22210723.7
Mar. 15, 2023 (EP) 23162124.4

(51) Int. Cl. A61K 47/64; A61K 47/55

(52) U.S. Cl. CPC A61K 47/64; A61K 47/55

(57) ABSTRACT

The use of a compound A as a peptide binder specifically binding to the ubiquitin ligase adapter FBXO31, the compound A having a terminal binding region of the general formula (i), U—NH₂ (i), with U being of the formula (ii), [NR—V—CO]ₙ-(ii), in which V is an organic moiety, and R is hydrogen, a linear or branched, saturated or unsaturated C₁ to C₁₀ alkyl residue, or forms together with a residue of V a ring system,

wherein U is a sequence of three amino acids and the C-terminal amino acid of U is selected from the group consisting of I, L, V, F, N, Y, S, T, A, W and M.

### REFERENCE TO AN ELECTRONIC SEQUENCE LISTING

The present application contains a Sequence Listing that has been submitted electronically and is hereby incorporated by reference in its entirety. The electronic Sequence Listing is named Substitute_Sequence_Listing_ST26, was created on Jun. 12, 2026, and is 80,977 bytes in size.

The present invention relates to a compound (in the following referred to as compound A) for use as a peptide binder specifically binding to the ubiquitin ligase adapter FBXO31 in a human or animal body according to the preamble of claim 1. In particular, the invention relates to compound A for use as a therapeutic agent to induce selective intracellular degradation of a disease-associated protein as well as to a conjugate or multimer comprising a protein P linked to compound A and containing a binding domain or structure capable of binding specifically to a disease-associated protein.

Cellular protein homeostasis describes the essential process of regulating the function, localization and turnover of proteins. At the molecular level, this is often achieved by post-translational modifications that either directly control protein function or mark them for further processing by downstream effectors. While most well-studied post-translational modifications are deposited or erased by dedicated enzymes, amino acid side chains and the protein backbone itself can also experience a plethora of non-enzymatic modifications, such as oxidative damage or alkylation.

Selective protein degradation is typically initiated by a substrate receptor that recognizes its client protein via a characteristic sequence motif on the client, a so-called degron. The presence of a degron recruits the client for degradation by the general proteolytic machinery. Most prominently, ubiquitin ligases recognize a client degron and then modify the client via conjugation of the small protein tag ubiquitin onto lysine side chains. This is referred to as poly-ubiquitylation of a protein, which typically results in the recruitment and activation of the proteasomal complex for processive proteolysis. Specificity of this system is established by over 600 human ubiquitin ligases which can bind the specific degrons on their respective client proteins.

For example, the ubiquitination of proteins destined for proteasomal degradation can be catalyzed via the SCF complex, which is a multi-protein E3 ligase complex of Skp, Cullin, an F-box protein (FBP) and RBX1. Among these, FBP contributes to the substrate specificity of the SCF complex by interacting with a ubiquitination target protein through a protein interaction domain. FBP also binds to Skp1 of the SCF complex using F-box motif, bringing the target protein into proximity with the functional E2 ubiquitin-conjugating enzyme.

In addition to its role in cell physiology, selective protein degradation forms the basis for the emerging class of targeted protein degradation (TPD) therapeutics, which recruit disease-relevant proteins to the cellular proteolysis machinery for their subsequent degradation (Burslem, G. M. and Crews, C. M. (2020) ‘Proteolysis-Targeting Chimeras as Therapeutics and Tools for Biological Discovery’, Cell. Elsevier Inc., 181 (1), pp. 102-114; Hanzl, A. and Winter, G. E. (2020) ‘Targeted protein degradation: current and future challenges’, Current Opinion in Chemical Biology. Elsevier Ltd, 56, pp. 35-41). Specifically, TPD aims at targeting a wide range of previously intractable proteins such as oncogenic transcription factors or neurotoxic aggregates.

For example, so-called PROTACs (proteolysis-targeting chimera) are currently being developed (see for example Nature Biotechnology, Vol. 40, January 2022, pp. 12-16). PROTACs are heterobifunctional small molecules, one moiety of which binds to an E3 ubiquitin ligase, and the other end, joined by a linker, attaches to the protein target of interest.

Systematic assessments of TPD-inducing probes showed that currently exploited E3 ligases have very different target preferences, even if tethered to the same protein (Donovan, K. A. et al. (2020) ‘Mapping the Degradable Kinome Provides a Resource for Expedited Degrader Development’, Cell. Elsevier Inc., 0(0), pp. 1714-1731). New E3 ligase ligands are therefore required to modulate the selectivity of existing drug candidates and extend the target scope of TPD therapeutics.

In the past, efforts have thus been made to elucidate the underlying concepts mediating selective protein degradation in more detail and to explore chemical modifications which can lead to the molecule to bind to E3 ligases. However, whether any of the modifications proposed so far are indeed sufficient to mark proteins for degradation and how they are read by the proteostasis machinery, remains unknown.

In consideration of the above, the problem of the present invention is therefore to provide a peptide binder which specifically binds to an E3 ligase or any of its subunits while allowing a high degree of flexibility in the design of the peptide binder. Specially, a peptide binder shall be provided which can be tailored to mark a wide variety of disease-associated proteins selective intracellular degradation. More specifically, a peptide binder for the therapeutic use as a degrader or a molecular glue of disease-associated proteins shall be provided.

In the specific case of the design of the peptide binder starting from a peptide backbone, the present invention shall allow to mark the peptide backbone for specific FBXO31 binding by means of a relatively simple modification.

The problem is solved by the subject matter according to claim 1. Further preferred embodiments are subject of the dependent claims.

According to claim 1, the present invention relates to a compound A for use as a peptide binder specifically binding to the ubiquitin ligase adapter FBXO31 in a human or animal body, the compound A having a terminal binding region of the general formula (i)

—U—NH₂ (i)

with U being of the formula (ii)

—[NR—V—CO]ₙ (ii),

in which V is an organic moiety, and
R is hydrogen, a linear or branched, saturated or unsaturated C₁ to C₁₀ alkyl residue, or forms together with a residue of V a ring system,
wherein U is a sequence of three amino acids and the C-terminal amino acid of U is selected from the group consisting of I, L, V, F, N, Y, S, T, A, W and M.

The group, of which the C-terminal amino acid is selected from, thereby includes both the canonical amino acids as well as unnatural derivates thereof.

In the context of the present invention, it has surprisingly been found that by providing a peptide binding region of the formula defined above, a selective binding to FBXO31 can be established, which forms the basis for recruiting the peptide binder or any protein linked to it to selective degradation.

In particular, it has been found that if the peptide binder is a peptide (and therefore U relates to a peptide backbone) amidation of its C-terminus is sufficient to recruit it for degradation via the ubiquitin-proteasome system. Thus, adding an amide to the C-terminus can be used as a universal tool to mark almost any terminal sequence to become a new FBXO31 substrate for degradation.

According to formulae (i) and (ii), U thus relates to an amino acid sequence, in which the terminal carboxy group of the C-terminal amino acid is amidated. In other words, the carboxy group at position C-alpha is amidated. Hence, a sequence containing an amide group in a position other than at its C-terminus does not fall under the definition of U unless its C-terminal carboxy group is amidated as well. In particular, Cyclin D1 which has been reported to be a binder for FBXO31 does not fall within the definition of of compound A according to the present invention, since its C-terminal carboxy group is not amidated.

In more general terms, the present disclosure also relates to a compound having a terminal binding region comprising the moiety U as defined by formula (ii) above, and being devoid of a terminal carboxy group.

In the context of the present invention, it has further been found that particularly strong binding to FBXO31 is achieved if U comprises a C-terminal amino acid of the group of amino acids defined in claim 1.

Owed to the ability of FBXO31 to bind a broad range of terminal motifs, the present invention allows for a high degree of flexibility in the development of peptide binders. Specifically, a broad variety of ligand regions (typically linked to the N-terminus) having the potential of binding to a multitude of different disease-associated proteins can be combined with the C-terminal FBXO31 binding region.

According to claim 1, the present invention relates to compound A defined above for use as a peptide binder specifically binding to FBXO31 in a human or animal body, and hence to a method for treatment of the human or animal body by therapy or a diagnostic method practiced on the human or animal body. Apart from the use of compound A as a peptide binder for a therapeutic or diagnostic method, the present disclosure further relates to the use of compound A for a diagnostic method outside the human or animal body.

As mentioned above, amidation of the C-terminus of the peptide backbone is sufficient to recruit it for the degradation via the ubiquitin-proteasome system. As will be pointed out in further detail below, this peptide binder can contain a ligand moiety having a binding domain or structure for the specific interaction with a disease-associated protein of interest, thus marking the latter for intracellular degradation. The ligand moiety can in particular be a peptide or a small molecule. Although the use of a relatively small ligand moiety is preferred, it is also thinkable to link the peptide binder to an antibody or a fragment thereof.

As also mentioned above, the design of the peptide binder starts from a peptide backbone, which can comprise canonical or non-canonical amino acids.

According to a very straightforward and thus preferred embodiment of the present invention, U is a sequence of canonical amino acids (with the C-terminal amino acid being amidated).

As will be discussed in further detail by way of the working examples, analysis of the amidated peptides binding most strongly to FBXO31 revealed a marked preference for aliphatic amino acids Ile, Leu and Val besides non-polar residues Asn and Tyr.

According to a preferred embodiment of the invention, the C-terminal amino acid of U is selected from the group consisting of I, L, V, F, N, Y, S, T and A. In this regard, it is particularly preferred that each of the three last C-terminal amino acids of U is selected from the group consisting of I, L, V, F, N and Y, in particular of I, L, V, F.

According to a further preferred embodiment, U contains a C-terminal motif of the amino acid sequence x₁x₂x₃, with
x₁ being selected from the group consisting of V, D, S and A;
x₂ being selected from the group consisting of I, V and T; and
x₃ being selected from the group consisting of I, L, V and F.

In this regard, it is particularly preferred that U contains a C-terminal motif of the sequence SVV, as this was found to be the top-scoring terminus in FBXO31-binding assays. Fluorescence polarization spectroscopy confirmed that the SVV terminus shows high affinity for FBXO31 only in its amidated form.

In view of its intended use to be linked to a ligand moiety (in particular a peptidic or small molecule ligand moiety), which specifically binds to a disease-associated protein (and thus recruits the disease-associated protein for degradation), compound A preferably contains a linker peptide. Specifically, compound A is according to this preferred embodiment of the general formula (iii)

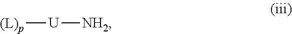

with L being a linker peptide comprising two or more natural amino acids and p being 0 or 1. More preferably, the linker peptide L comprises at least 3 amino acids.

According to a further aspect, compound A is of general formula (iv),

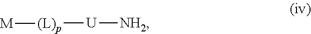

wherein M is a spacer moiety selected from the group consisting of linear or branched (C₁-C₂₀)-alkylene, linear or branched (C₂-C₂₀)-alkenylene, linear or branched (C₂-C₂₀)-alkynylene, (C₃-C₂₀)-cycloalkylene, or any combination thereof, wherein one or more carbon atoms in said groups is optionally replaced with a heteroatom selected from O, S, N, NH or N(C₁-C₆)-alkyl.

The spacer moiety allows to separate the C-terminal end of U further from the binding domain or structure capable of binding specifically to a disease-associated protein.

Preferably, the spacer moiety M is a linear (C₁-C₁₀)-alkylene chain or PEG with 1 to 5 repeating units (—O—CH₂—CH₂—), since they are relatively inert toward biological targets. Moreover PEG-linkers have a good aqueous solubility.

According to a further aspect, the present invention also relates to compound A, i.e. the peptide binder described above, for the use as a therapeutic agent to induce selective intracellular degradation of a disease-associated protein. In this regard, a preferred embodiment relates to compound A for the use as a heterobifunctional degrader or a monovalent molecular glue.

As already mentioned before, amidation of a protein's C-terminus as defined above is sufficient to mark proteins for selective degradation via the SCF^{FBXO31} complex, and hence allows for an efficient clearance of a disease-associated protein by binding to the latter.

Besides the potential of the peptide binder itself functioning as a degrader, it can also be linked to a ligand moiety, which contains a binding domain or structure capable of binding specifically to a disease-associated protein.

Specifically, the ligand moiety can be peptide or a small molecule.

According to a still further aspect, the present invention additionally thus relates to a conjugate or multimer comprising
a) compound A as defined in any of the preceding claims and
b) a ligand moiety linked to compound A and containing a binding domain or structure capable of binding specifically to a disease-associated protein.

In this regard, it is of particular interest that the disease-associated protein to be specifically bound by the binding domain or structure of the ligand moiety is selected from the group consisting of transcriptional regulators, oncogenic proteins and neurotoxic proteins, especially those lacking otherwise targetable enzymatic functions. In other words, the ligand moiety preferably comprises a binding structure or domain specifically binding to a transcriptional regulator, an oncogenic protein or a neurotoxic protein.

Although the use of a small ligand moiety is preferred, the peptide binder can also be linked to a protein P, which contains a binding domain or structure capable of binding specifically to a disease-associated protein, thus together with protein P forming a protein conjugate functioning as the degrader for the disease-associated protein.

For example, protein P can be selected from the group consisting of an antibody or a fragment thereof, specifically a Fab fragment or F(ab′)₂ fragment, containing a complementary-determining region specific for the disease-associated protein forming the antigen.

As will also be explained in further detail by way of the specific working examples, the protein conjugate or multimer can be prepared by a method comprising the step of chemoenzymatically conjugating to the C-terminus of protein P a compound of formula (iv)

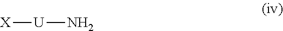

wherein X is a peptide comprising two or more natural amino acids and
U is defined as above.

Specifically, the chemoenzymatic conjugation can thereby be performed by using sortase. To this end, protein P contains at its C-terminus a C-terminal sortase recognition motif, and peptide X containing at its N-terminus an N-terminal sortase recognition motif.

More specifically, the C-terminal sortase recognition motif is a sortase A recognition motif, in particular LPXTG (A-MO1; SEQ ID NO: 1) or LPETGG (A-MO2; SEQ ID NO: 2), with X being any amino acid, or a sortase B recognition motif, in particular NPQTN (B-MO1; SEQ ID NO: 3) or NPKTG (B-MO2; SEQ ID NO: 4).

In particular, the N-terminal sortase recognition motif consists of three glycine-serine repeats (GS-MO; SEQ ID NO: 5) or four glycine-serine repeats (GS-MO2; SEQ ID NO: 6), or of more than two glycine residues, in particular of three to ten glycine residues, more particularly of three glycine residues (GGG), four glycine residues or five glycine residues GGGGG (G-MO1; SEQ ID NO: 7).

| | | |
|---|---|---|
| Name | SEQ ID NO.: | Sequence |

sortase recognition motif

| | | |
|---|---|---|
| A-MO1 | 1 | LPXTG |
| A-MO2 | 2 | LPETGG |
| B-MO1 | 3 | NPQTN |
| B-MO2 | 4 | NPKTG |
| GS-MO | 5 | GSGSGS |
| MS-Mo2 | 6 | GSGSGSGS |
| G-MO1 | 7 | GGGGG |

Depending on the specific application and purpose, other methods of preparing the protein conjugate or multimer can be used.

Within the context of the present invention “alkyl” means a straight or branched chain unsubstituted hydrocarbon group, preferably comprising 1 to 20 carbon atoms.

The term heteroalkyl refers to an alkyl, alkenyl or alkynyl group as defined herein, where one or more and preferably 1, 2 or 3 carbon atoms are replaced independently of each other by an oxygen, nitrogen, phosphorous or sulphur atom, for example an alkoxy group containing from 1 to 10 carbon atoms, preferably from 1 to 6 carbon atoms, e.g. 1 to 4 carbon atoms, such as methoxy, ethoxy, propoxy, iso-propoxy, butoxy or tert.-butoxy; a (1-4 C)alkoxy(1-4° C.)alkyl group such as methoxymethyl, ethoxymethyl, 1-methoxyethyl, 1-ethoxyethyl, 2-methoxyethyl or 2-ethoxyethyl; or a cyano group; or a 2,3-dioxyethyl group.

Within the context of the present invention, “substituted alkyl” means an alkyl group substituted with one to four substituents selected from the group consisting of fluoro, chloro, bromo, iodo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, —SO₂NH₂, substituted sulfonamido, nitro, cyano, carboxy, —CHO, —CH(COOH)₂, CH(CONH₂)₂, —CH(COOalkyl)₂, —CONH₂, —CONHalkyl, —CONHaryl, CONHaralkyl, —NHCOalkyl, —NHCOaryl, —NHCOaralkyl, alkoxycarbonyl, guanidine. Preferably, a substituted alkyl is selected from the group consisting of a linear alkyl substituted by hydroxy, CHO, carboxy, CH(COOH)₂, —NHCOalkyl, mercapto, imidazolyl, methylthio, aryl, amino, guanidine, CHO, and —CH(COOH)₂.

Within the context of the present invention, “alkenyl” means a straight or branched chain unsubstituted hydrocarbon group which contains at least one double bond, preferably comprising 1 to 20 carbon atoms.

Within the context of the present invention “substituted alkenyl” means an alkenyl group substituted with one to four substituents. The substituents include one to four substituents as recited above as alkyl substituents.

Within the context of the present invention “alkynyl” means a straight or branched chain unsubstituted hydrocarbon group which contains at least one triple bond, preferably comprising 1 to 20 carbon atoms.

Within the context of the present invention “substituted alkynyl” means an alkynyl group substituted with one to four substituents. The substituents include one to four substituents as recited above as alkyl substituents.

Within the context of the present invention, “cycloalkyl” means an optionally substituted, saturated cyclic hydrocarbon ring systems containing 1 to 3 rings and 3 to 7 carbons per ring which may be further fused with one or more heterocycloalkyl, aryl or heteroaryl groups, wherein when substituted, the substituents will include one or more substituents as recited above as alkyl substituents.

Within the context of the present invention, “aryl” means a monocyclic or bicyclic aromatic hydrocarbon group having 6 to 12 carbon atoms in the ring portion.

Within the context of the present invention, “substituted aryl” means an aryl group substituted by one to four substituents selected from alkyl, substituted alkyl, halo, trifluoromethoxy, trifluoromethyl, hydroxy, alkoxy, cycloalkyloxy, heterocyclooxy, alkanoyl, alkanoyloxy, amino, alkylamino, aralkylamino, cycloalkylamino, heterocycloamino, dialkylamino, alkanoylamino, thiol, alkylthio, cycloalkylthio, heterocyclothio, ureido, nitro, cyano, carboxy, carboxyalkyl, carbamyl, alkoxycarbonyl, alkylthiono, arylthiono, alkysulfonyl, sulfonamido, and aryloxy.

Within the context of the present invention, “heterocyclo” means an optionally substituted, fully saturated or unsaturated, aromatic or nonaromatic cyclic group, which is a 4 to 7 membered monocyclic, 7 to 11 membered bicyclic, or 10 to 15 membered tricyclic ring system, having at least one heteroatom in at least one carbon atom-containing ring, each ring of the heterocyclic group containing a heteroatom may have 1, 2 or 3 heteroatoms, wherein the term “heteroatoms” shall include oxygen, sulfur and nitrogen; and wherein when substituted, the substituted heterocyclo group will include one or more substituents as recited above as alkyl substituents, preferably hydroxy, alkylhydroxy, amino, nitro, fluoro, chloro, bromo, iodo and CHO.

Within the context of the present invention, “aralkyl” means a radical —RₐR_{b} where Rₐ is an alkylene group and R_{b} is an aryl group as defined herein, e.g., benzyl, phenylethyl and the like.

Within the context of the present invention “aralkenyl” means a radical —RₐR_{b} where Rₐ is an alkenylene group and R_{b} is an aryl group as defined herein, e.g., 3-phenyl-2-propenyl, and the like.

Within the context of the present invention, “heteroaralkyl” means an alkyl group substituted with a heterocyclic ring as defined above.

Within the context of the present invention, “heteroaralkenyl” means an alkenyl group substituted with a heterocyclic ring as defined above.

### EXAMPLES

The present invention is further illustrated by way of the working examples, which are described in the following.

### Design of Experiments

As will be pointed out in detail further down below, the effect of selected chemical modifications on protein turnover was studied by generating a set of semi-synthetic fluorescent reporter proteins that display individual modified amino acids in a flexible C-terminal peptide tail. This was achieved by recombinant production of super-folder green fluorescent protein carrying an acceptor sequence for the transpeptidase sortase A. Using solid phase peptide synthesis (SPPS), peptides containing modified amino acids that mimic common forms of protein damage were generated; tyrosine modification through oxidation or misincorporation (L-3,4-dihydroxyphenylalanine, L-DOPA), advanced glycation end products (N(6)-carboxymethyllysine), carbamylation (homocitrulline) and enzymatic or spontaneous backbone cleavage through alpha amidation (C-terminal amidation, CTA). Conjugation with sortase A under optimized conditions, yielded a set of highly pure modified proteins for their subsequent study in human cells.

The effect of the C-terminal amidation on protein degradation was measured in the human erythroleukemia cell line K562, as will also be pointed out by way of the specific working examples.

To identify cellular machinery underlying the recognition and removal of C-terminally amidated proteins (CTAPs), a genome-wide CRISPR screen for genes that are required for degradation of C-terminally amidated GFP was devised. Since knockout of central protein quality control and -turnover genes is not tolerated, a clonal K562 cell line with a tightly controllable Cas9 allele (iCas9) was generated. Following transduction with a previously developed genome-wide sgRNA library, knockout was induced for 5 days to allow for efficient knockout while minimizing loss of cells with defects in essential pathways. Next, a C-terminally amidated GFP variant was delivered by nucleofection together with an internal control protein. Following the onset of protein degradation, cells that were proficient in general protein turnover but showed a specific defect in CTAP-clearance (BFP⁻GFP⁺) were isolated, as well as an unaffected control population (BFP⁻GFP⁻). Deep sequencing of sgRNA-vectors revealed a stark enrichment of few sgRNAs in the CTAP clearance deficient population, targeting the E3 ubiquitin ligase substrate receptor FBXO31. To validate its role in CTAP clearance in an orthogonal assay, FBXO31 was knocked down using SpCas9 fused to a transcriptional repressor (CRISPRi) and guide RNAs directing the repressor to FBXO31's transcription start site, and degradation of an independently amidated GFP conjugate was measured. FBXO31-targeting sgRNAs could completely stabilize the amidated reporter, while the unrelated RxxGxx-degron remained unaffected. These findings establish FBXO31 as central CTAP clearance factor.

In addition to FBXO31, the screen identified its known interactor, the SCF-type cullin ring ligase scaffold protein CUL1. Cullin ring ligase function depends on the activity of the COP9 signalosome complex, of which several subunits also scored highly (GPS1, COPS3, COPS5, COPS6, COPS8), suggesting that an FBXO31-containing SCF ligase assembly could ubiquitylate CTAPS for subsequent proteasomal removal. To test this model, HA-tagged FBXO31 was expressed in FBXO31 knockout cells and co-immunoprecipitation (co-IP) was performed. It was found that FBXO31 co-purified with core SCF components CUL1 and SKP1. Co-delivered semi-synthetic mCherry co-purified only in its C-terminally amidated form, supporting that SCF^{FBXO31} directly binds its amidated substrates in cellulo. To test whether FBXO31 directly binds amidated C-termini, recombinant SKP1/FBXO31 was generated and its affinity for fluorescently labeled peptides was measured in vitro by fluorescence polarization (FP) spectroscopy. While an amidated model peptide showed high affinity for SKP1/FBXO31 (K_{D}=22 nM), no interaction was observed for the non-amidated peptide. Similarly, FBXO31 displayed high affinity for the C-terminal recognition domain of the FBXO31 client Cyclin D1 in a strictly carboxamide-dependent manner (K_{D}=59 nM). It can thus be concluded that SCF^{FBXO31} recognizes CTAPs through direct binding of amidated C-termini.

To identify the preference of FBXO31 for C-terminal amino acids, fluorescein-modified peptides were prepared in which the C-terminal amino acid was varied to any of the twenty proteinogenic amino acids, and the binding affinity of FBXO31 to these amide-peptides was determined by fluorescence polarization measurement. FBXO31 bound to all peptides with a marked preference for hydrophobic amino acids. With Phe the peptide bound with a K_{D} of 6 nM. Charged amino acids bound with a markedly reduced affinity. Aspartic acid peptide being the weakest binder bound with an affinity of 370 nM.

In order to assess how FBXO31 recognizes CTAPs with such high selectivity, a pooled interaction profiling approach was employed to identify rules of C-terminus recognition by FBXO31. TO this end, pooled peptide libraries were synthesized by random incorporation of amino acids in the three C-terminal positions and FBXO31-binding was assayed for each in presence and absence of a terminal amide. SPPS with isokinetic mixtures of all proteinogenic amino acids except Cys yielded a library with 6,859 peptides. To measure binding of each peptide by FBXO31, in vitro co-IP of recombinant FBXO31/SKP1 were performed with each library and the abundance of FBXO31-bound peptides relative to an isobarically labeled input sample was quantified. co-IP of the non-amidated library could not identify any strongly FBXO31-bound peptides, suggesting that terminal Asn- or Gln FBXO31-side-chains cannot mediate FBXO31 binding in lieu of an amidated C-terminus. In contrast, co-IP of the amidated peptide pool identified 133 strongly bound C-terminal motifs (≥20% of input). Analyzing the composition of the top 100-bound termini, revealed a marked preference for aliphatic amino acids Ile/Leu and Val besides non-polar residues Asn and Tyr, while largely excluding the carboxylic acid containing Glu and Asp. Fluorescence polarization spectroscopy confirmed that the top-scoring terminus (Ser-Val-Val) shows high affinity for FBXO31 only in its amidated form. Recognition is therefore not solely based on hydrophobicity, but is highly specific for the terminal modification.

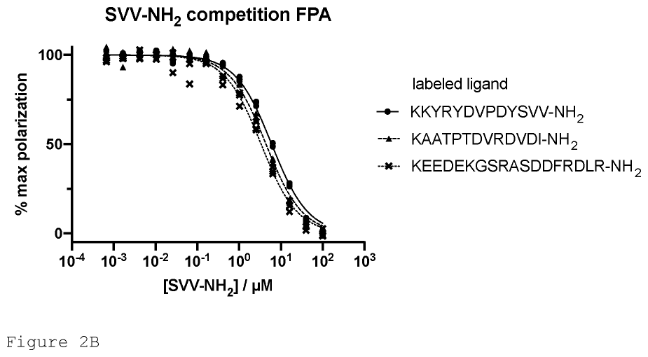

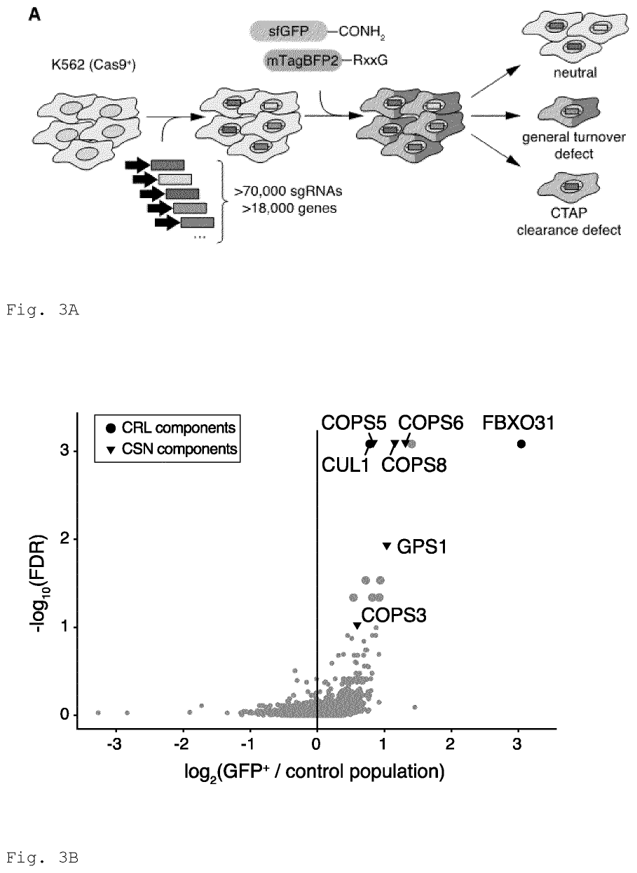

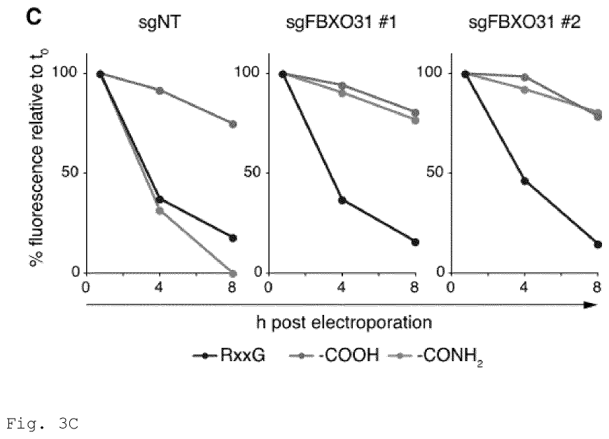

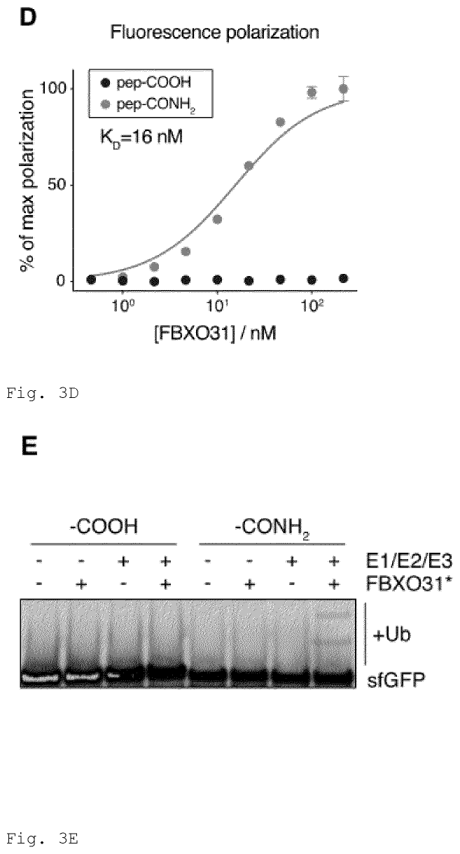

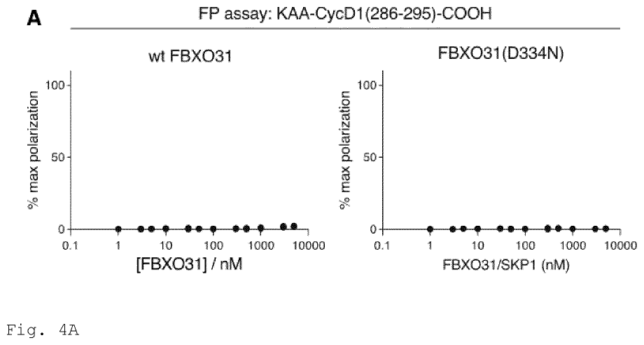

To test whether a lower molecular weight probe could be derived based on these rules, the C-terminally amidated tripeptide Ser-Val-Val-NH₂ (302.37 g/mol) was tested. In competitive fluorescence polarization anisotropy measurements this ligand could efficiently displace different fluorescent peptide probes from FBXO31 (FIG. 2B).

C-terminal amide bearing peptides can be readily converted into PROTACs. Using standard amide bond coupling conditions, the BET bromodomain ligand JQ1-acid of formula X

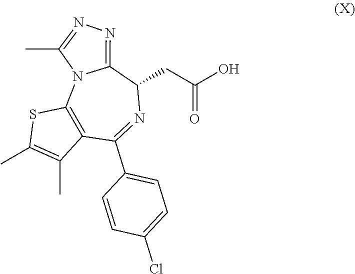 [SMILES: Cc1sc2c(c1C)C(c1ccc(Cl)cc1)=N[C@@H](CC(=O)O)c1nnc(C)n1-2]

was successfully attached to a peptide with a C-terminal amide. JQ1 as substrate recruiting moiety was either attached directly to the N-terminus of the peptide resulting in conjugate (XI; SEQ ID NO: 63)

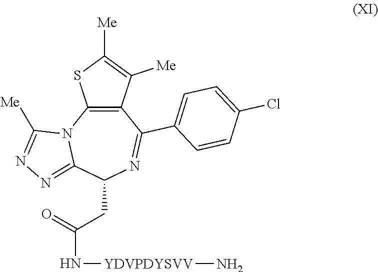

or attached to a linker which in turn was linked to the peptide's N-terminus resulting in conjugate XII (SEQ ID NO: 64) and conjugate XIII (SEQ ID NO: 65)

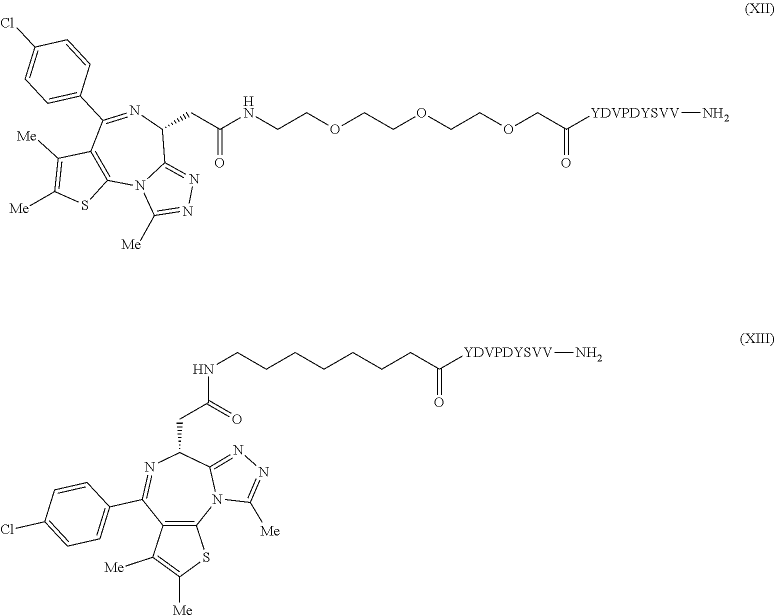

### General Materials and Methods

In the following, the general materials and methods of the working examples are outlined with referring to the figures attached.

### Identification of the Underlying Machinery

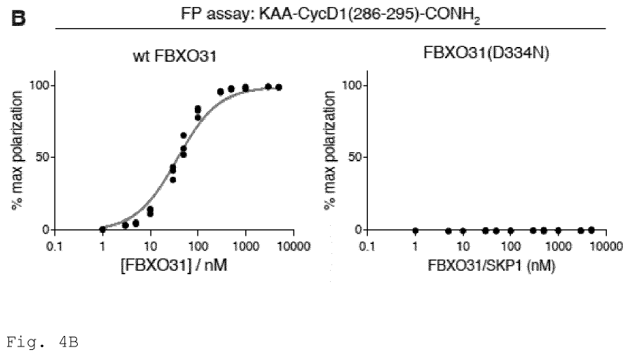

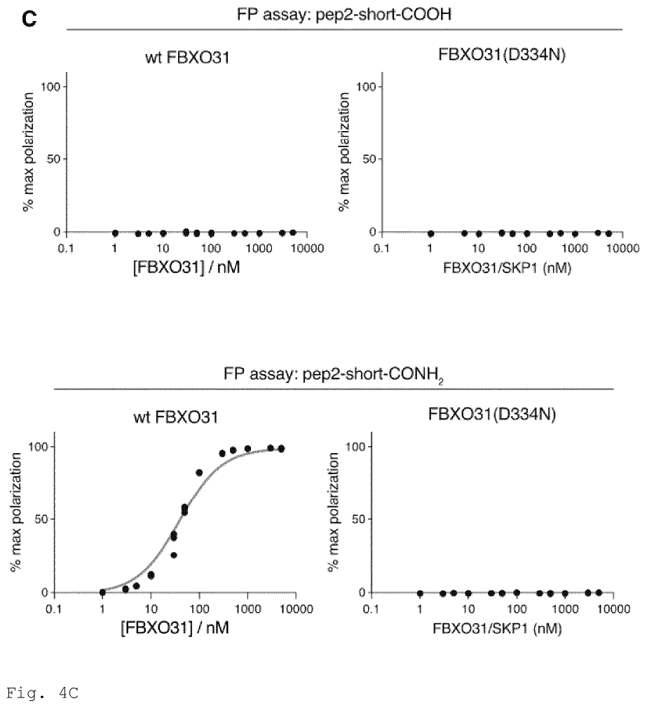

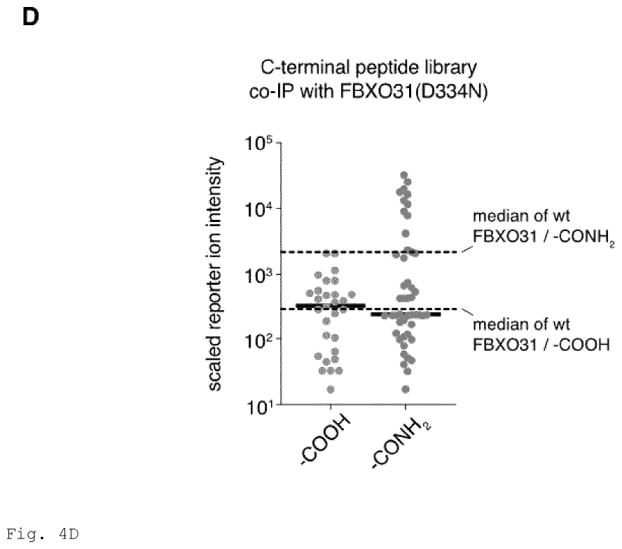

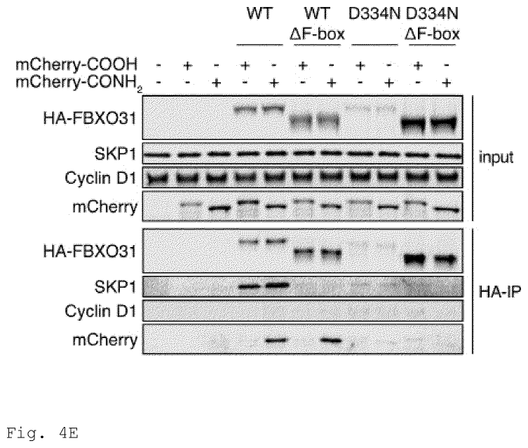

To identify the cellular machinery underlying the recognition and removal of C-terminally amidated proteins (CTAPs), a genome-wide CRISPR screen for genes responsible for specific degradation of C-terminally amidated sfGFP (sfGFP—CONH₂) was devised, as discussed above. The concept is illustrated in FIG. 3A. Since knockout of central protein quality control and -turnover genes may impede cell survival, a clonal K562 cell line with a tightly controllable Cas9 allele (iCas9) was generated. Following transduction with a previously developed TKOv3 genome-wide sgRNA library, Cas9 expression was induced for 5 days to allow for efficient knockout while minimizing loss of cells with defects in essential pathways. Next, a C-terminally amidated GFP variant was delivered by electroporation together with mTagBFP2-RxxG, an internal control protein for general protein turnover carrying a sequence-based degron. Following the onset of protein degradation (14 h), cells that were proficient in general protein turnover but no longer cleared the CTAP (BFP⁻GFP⁺) were isolated, as well as a control population (BFP⁻GFP⁻).

Bioinformatic analysis revealed a stark enrichment of few targeted genes in the CTAP clearance-deficient population. As shown in FIG. 3B, the most prominent hit was FBXO31, which is a substrate adaptor for the SCF (SKP1-CUL1-F-box protein) E3 ubiquitin ligase assembly.

### Validation of the Role of FBXO31 in CTAP Clearance

### Knock-Down

To test whether SCF/FBXO31 mediates CTAP clearance in an orthogonal assay, FBXO31 was knocked down using CRISPR inhibition (CRISPRi) and the degradation of an sfGFP conjugate with a different C-terminal sequence was measured. The results are given in FIG. 3C, which shows that FBXO31-targeting sgRNAs completely stabilized the amide-form of this reporter, while a reporter carrying the RxxG degron remained unaffected.

### Fluorescence Polarization

It was then tested whether SCF/FBXO31 directly binds and ubiquitylates amidated clients, or whether it plays an indirect role in CTAP clearance. To this end, recombinant FBXO31 in complex with the binding partner SKP1 was purified and its affinity for various peptides was measured by fluorescence polarization (FP). In vitro, FBXO31 bound the peptide used for screening with high affinity (K_{D}=16±2 nM) while no binding could be detected for its carboxylic acid form, as shown in FIG. 3D.

### Reconstitution of Ligase Assembly

To test whether FBXO31 binding leads to productive ubiquitylation of substrates, the full SCF/FBXO31 E3 ligase assembly was reconstituted from recombinant components. Indeed, the SCF/FBXO31 complex ubiquitylated sfGFP—CONH₂ in vitro, but had no detectable activity on sfGFP—COOH, as shown FIG. 3E. Together these results establish that SCF/FBXO31 is a reader of C-terminal protein amides and that this amide is required for SCF/FBXO31 to ubiquitylate targets.

### Alteration of Substrate Specificity by Cerebral-Palsy Associated Mutation

Based on the finding of a dominant de novo D334N mutation in FBXO31 among patients with diplegic spastic cerebral palsy, this mutation was speculated to act by increased degradation of Cyclin D1 and eliminates the negative charge promoting CTAP recognition. It was therefore assessed whether the D334N mutation alters FBXO31 substrate recognition and CTAP40 clearance.

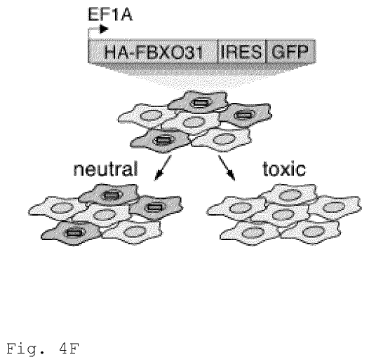

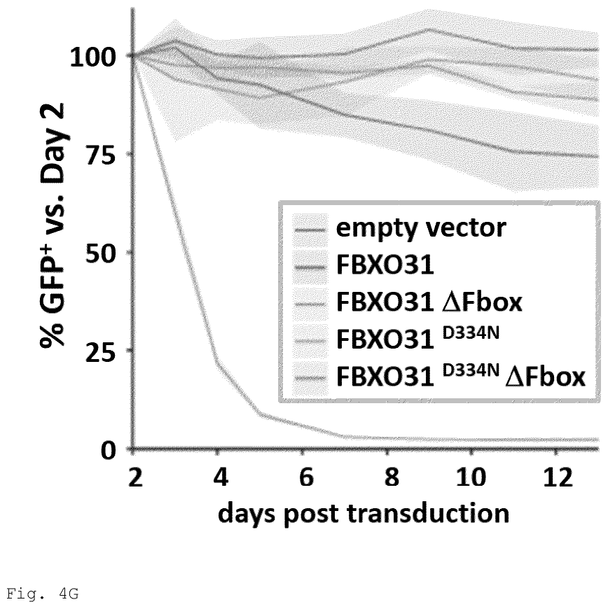

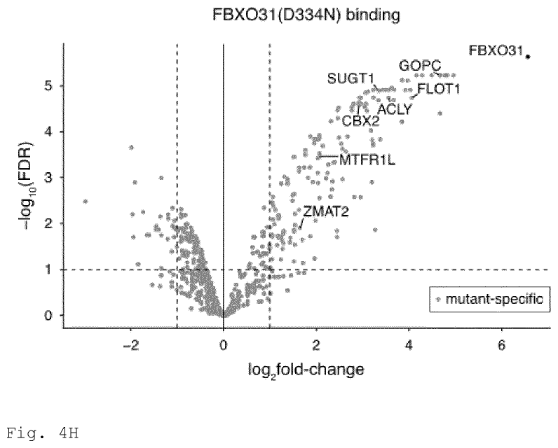

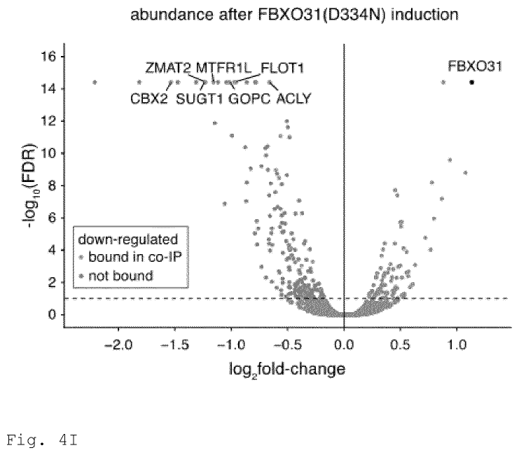

In vitro, neither wild type nor D334N mutant FBXO31 showed any affinity for the proposed C-terminal degron of Cyclin D1, as shown in FIG. 4A. However, the D334N mutation abolished binding to C-terminal amide peptides (FIGS. 4B and C). This also held true in a pooled peptide interaction screen covering >1200 peptides, where the D334N mutation displayed globally reduced CTAP binding (FIG. 4D). Likewise, FBXO31 (D334N, ΔF-box) expressed in FBXO31 knockout cells failed to immuno-precipitate both a model CTAP (mCherry-CONH₂) and unmodified Cyclin D1, as shown in FIG. 4E.

Based on the finding that full-length FBXO31 (D334N) could not be stably expressed over extended culture periods, even in cells expressing wildtype FBXO31, a competitive growth assay was performed to quantitatively test whether FBXO31 (D334N) compromises cell survival, the results of which being shown in FIG. 4F. While wildtype FBXO31 CDNA expression was well tolerated in FBXO31 knockout HEK293T cells, the D334N mutant was rapidly depleted from co-culture, as shown in FIG. 4G. Deletion of the F-box motif required for SCF complex assembly fully abolished this effect, suggesting that FBXO31 (D334N) exerts a toxic ubiquitin ligase activity.

Co-IP MS of FBXO31(ΔF-box) was performed using both the wildtype and D334N mutant to determine how the mutation alters substrate recognition. FBXO31 (D334N, ΔF-box) formed detectable interactions with 220 proteins, 195 of which were for the wildtype (FIG. 4H). It was tested not detected whether these putative neo-substrates are down-regulated in response to acute FBXO31 (D334N) expression using the ligand-inducible shield-degron system. Tandem mass tag (TMT) expression proteomics identified a marked reduction in the abundance of several candidates within 12 h of inducing DD-FBXO31 (D334N), but not the wildtype (FIG. 4I). Among these neo-substrates were core-essential proteins (ACLY, SUGT1 and PRDX2), which could account for the observed proliferation defect. Based on these results it can be concluded that D334 is required for CTAP binding, and the cerebral-palsy associated mutation is dominant because it redirects ubiquitin ligase activity away from C-terminal amide substrates and towards multiple essential cellular proteins.

### Characterization of Binding of FBXO31 to CTAP

To determine the substrate scope of FBXO31, in vitro binding studies as discussed in the following were carried out:

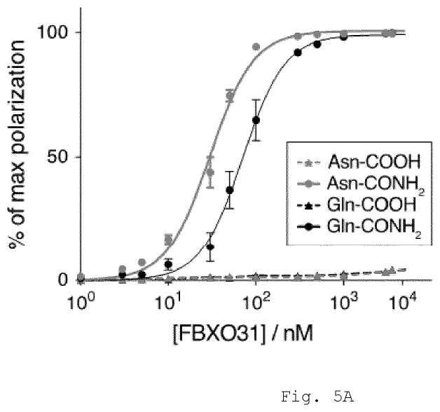

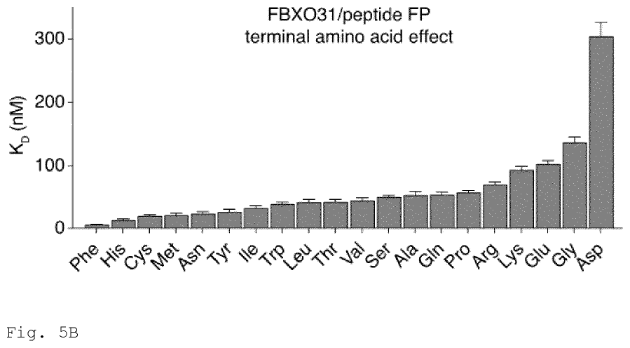

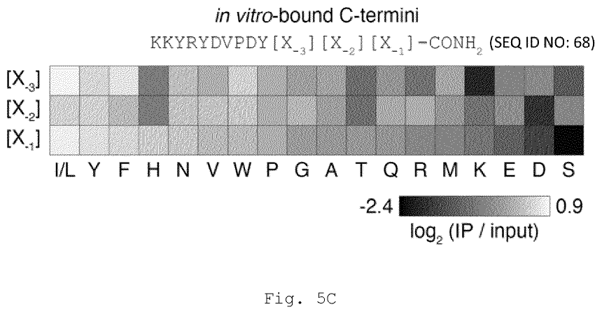

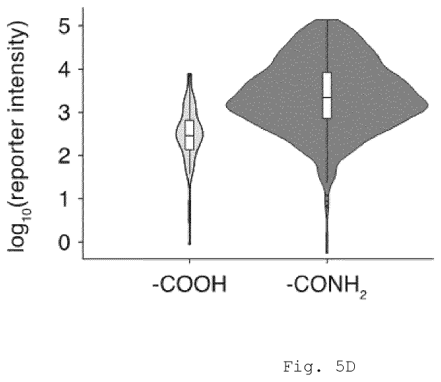

In this regard, it was first tested whether FBXO31 specifically binds C-terminal amides rather than side chain amides in asparagine or glutamine. FP assays using recombinant FBXO31/SKP1 and fluorescently labeled peptides showed no affinity for peptides with unmodified N or Q at the C-terminus. However, the same peptide sequences were bound with high affinity when carrying a C-terminal primary amide (X—N—CONH₂: K_{D}=24±3 nM, X-Q-CONH₂: K_{D}=55±4 nM), as shown in FIG. 5A. Extending this assay to peptides bearing the primary amide derivatives of each of the 20 natural amino acids revealed that FBXO31 can bind virtually any C-terminal amide with nanomolar affinity although affinities for the specific amino acids differ slightly, as shown in FIGS. 5B and 5C. The weakest binders were peptides bearing glycine and acidic residues, with X-D-CONH₂ exhibiting a K_{D} of 304±22 nM. Hydrophobic residues were most strongly bound, with X—F—CONH₂ as the best substrate (KD≈6 nM), followed by other and then uncharged and charged hydrophilic side chains. These findings demonstrate that FBXO31 binds diverse C-terminal amides with high affinity and selectivity over unmodified C-termini and side chain amides.

Individual testing of amidated C-termini was extended by devising a massively parallel protein-peptide interaction screen to identify rules of the broader substrate preference by FBXO31. Using isokinetic mixtures of 19 natural amino acids (all except cysteine) in the first three coupling steps, peptide libraries containing >2′000 distinct C-termini detectable by mass spectrometry (MS) were synthesized. To quantify FBXO31/SKP1 binding to these sequences, in vitro co-immunoprecipitation of the library was performed and the abundance of each peptide alongside the input pool was quantified using isobaric labeling and MS. Overall, 841 distinct C-terminal amides co-purified with FBXO31, compared to just 73 unmodified C-termini. In addition, the C-terminal amides also showed 7.6-fold greater enrichment compared to unmodified peptides (FIG. 5D). Compared to the input library, FBXO31-bound peptides were enriched for hydrophobic side chains, while acidic residues were disfavored, especially in the terminal position. Despite these preferences, each tested amino acid could be detected in any of the three ultimate positions among bound peptides. The overall conclusion was drawn that FBXO31 is specific for peptide amidation and disfavors negatively charged termini. Unlike conventional sequence-based C-degrons FBXO31 is rather agnostic to specific sequence motifs, potentially enabling it to broadly surveil C-terminal amides across the diverse proteome.

The specific methods are described in the following in more detail:

### Reagents and Solvents

Fmoc-amino acids with suitable side-chain protecting groups, HATU (1-[bis(dimethylamino)methylene]-1H-1,2,3-triazolo[4,5-b]pyridinium 3-oxide hexafluorophosphate) were purchased from Peptides International (Louisville, KY, USA), ChemImpex (Wood Dale, IL, USA) and Merck Milipore. HPLC grade CH3CN from Sigma-Aldrich was used for analytical and preparative HPLC purification. Trifluoroacetic acid for HPLC analytical and preparative HPLC purification was purchased from ABCR. DMF (>99.8%) from Sigma-Aldrich and N-methylpyrrolidine from ABCR were directly used without further purification for solid phase peptide synthesis. Other commercially available reagents and solvents were purchased from Sigma-Aldrich (Buchs, Switzerland), Acros Organics (Geel, Belgium) and TCI Europe (Zwijndrecht, Belgium).

### Characterization

High-resolution mass spectra were recorded by the Molecular and Biomolecular Analysis Service (MoBiAS) at ETH Zurich with a Bruker maXis instrument (ESI-MS measurements) equipped with an ESI source and a Q-TOF detector. Reaction monitoring was performed on a Bruker microFLEX instrument (MALDI-TOF) using 4-hydroxy-a-cyanocinnamic acid as matrix.

LC-MS/MS analysis was performed on a timsTof pro (Bruker). Peptides were applied to a C18 reverse phase HPLC column and resolved using a binary buffer system of buffer (A) (0.1 vol % formic acid) and buffer B (acetonitrile, 0.1 vol % formic acid). A stepwise gradient starting with 5% B for 5 min followed by an increase to 95% B over 90 min was used to elute peptide. Peptides were recorded in a range of 300-2,000 m/z. Data was converted to .mgf file format using Peaks and analyzed using MASCOT. A custom database was generated containing the sequences of the two- and three-variable library. Error for ms1 was set to 10 ppm and for ms2 to 0.05 Da. False-discovery rate was estimated by decoy search and all peptide below FDR were excluded. For C-terminal amide libraries C-terminal amidation was set as a fixed modification.

### Purification

Peptides were analyzed and purified by reverse phase high performance liquid chromatography (RP-HPLC) on JASCO analytical and preparative instruments equipped with dial pump, mixed and in-line degasser, a variable wavelength UV detector (simultaneous detection of the eluent at 220 nm, 254 nm and 301 nm) and a Rheodyne injector with a 200 μL or 10 mL injection loop. Columns were heated to 60° C. using a Jetstream 2 column heater (analytical) or a H₂O water bath (preparative). The mobile-phase for RP-HPLC was Milipore-H2O containing 0.1% (v/v) TEA and HPLC grade CH3N containing 0.1% (v/v) TFA. Analytical HPLC was performed on Shiseido Capcell Pak C18 (5 μm, 4.6 mm I.D.×250 mm) columns at a flow rate of 1 mL/min. Preparative HPLC was performed on Shiseido Capcell Pak MGIII (5 μm, 20 mm I.D.×250 mm) at a flow rate of 10 mL/min.

### General Analytical HPLC Methods:

flow 1 mL/min, isocratic 10% CH₃CN for 3 min, then gradient from 10% to 95% CH₃CN in 14 min.

### General Preparative HPLC Methods:

flow 10 mL/min, isocratic 10% CH₃CN for 5 min, then gradient from 10% to 65% CH₃CN in 28 min.

### Solid Phase Peptide Synthesis

Loading of amino acids on solid support was performed as followed:

Chloro-trityl resin: The amino acid (1.20 equiv of desired loading) was dissolved in CH2C12 (200 mM). NMM (2 equiv) was added to the solution. The solution was given to preswollen chloro-trityl resin and shaken for 1 h. The resin was washed with CH2Cl2 and DMF. Remaining chloro-trityl moieties were capped with CH2Cl2/MeOH/NMM (17:2:1, v: v: v) for 1 min. The capping step was repeated once. The resin was washed with CH₂Cl₂ and DMF. The resin was dried using a N2 stream prior to usage.

Rink amide resin: Fmoc-Rink amide resin was deprotected using 20 vol % Piperidine in DMF for 2×5 min. The resin was washed thoroughly. Amino acid (1.2 equiv of desired loading) and HCTU (0.95 equiv of amino acid) were dissolved in DMF (200 mM). NMM (2 equiv of amino acid) was added. The solution was added to the preswollen Rink amide resin and shaken for 18 h. The resin was washed with CH₂C12 and DMF. The resin was dried using a N2 stream prior to usage.

Peptides were synthesized on a Multisyntech Syro I parallel synthesizer using Fmoc-SPPS chemistry.

### General Methods on Multisyntech Syro I Parallel Synthesizer:

Amino acids were dissolved in DMF to a concentration of 0.5 M. HATU was dissolved in DMF to a concentration of 0.5 M. DIPEA was dissolved in NMP to a concentration of 2 M. Amino acid, HATU and DIPEA are mixed to a final concentration of 0.2 M, 0.2 M and 0.4 M, respectively, and added to the resin.

The resin was agitated for 45 min. Coupling steps were repeated once.

Capping was performed with acetic anhydride. 20 vol % acetic anhydride in DMF was mixed with 2 M DIPEA at a ratio of 3:2 and added to the resin. The resin was agitated for 5 min. The capping step was repeated once.

Fmoc deprotection was performed with 20 vol % piperidine in DMF for 10 min. The deprotection step was repeated once.

### Solid Phase Peptide Synthesis

For fluorescein modified peptides, Boc-Lys(Fmoc)-OH (2 equiv) was coupled as the N-terminal residue using HATU. Fmoc was removed by treatment with 20 vol % piperidine. All following steps were performed in the dark. FITC (3 equiv) and NMM (6 equiv) were dissolved in DMF, given to the resin and shaken for 2 h. The resin was thoroughly washed using DCM and DMF. Peptide was cleaved form the resin using TFA/DODT/H2O (95:2.5:2.5, v/v) for 1 h. The resin was removed by filtration and the filtrate concentrated under reduced pressure. The solution was triturated with Et₂O and centrifuged to obtain crude peptide. The crude peptide was dissolved in H₂O/CH3N (1:1, v/v)+0.1% (v/v) TFA and purified using preparative HPLC.

The following peptides were synthesized:

| | | |
|---|---|---|
| Name | SEQ ID NO. | Sequence |

Probes for sortase conjugation

| | | |
|---|---|---|
| Pep1-DOPA | 8 | GGGKDLEGKGGSXGSGSXGGSKYPYDVPDYAKD-[OH], [X] = |
| | | 3,4-dihydroxyphenylalanine |
| Pep1-HNE | 9 | GGGKDLEGKGGSXGSGSXGGSKYPYDVPDYAKD-[OH], [X] = |
| | | 4-hydroxy-2-nonenal |
| Pep1-CML | 10 | GGGKDLEGKGGSXGSGSXGGSKYPYDVPDYAKD-[OH], [X] = |
| | | N(6)-carboxymethyllysine |
| Pep1-hCit | 11 | GGGKDLEGKGGSXGSGSXGGSKYPYDVPDYAKD-[OH], [X] = |
| | | homocitrulline |
| Pep1-Ala | 12 | GGGKDLEGKGGSAGSGSAGGSKYPYDVPDYAKD-[OH] |
| Pep1-Tyr | 13 | GGGKDLEGKGGSYGSGSYGGSKYPYDVPDYAKD-[OH] |
| Pep1-NH2 | 14 | GGGKDLEGKGGSAGSGSAGGSKYPYDVPDYAKD-[NH2] |
| Pep1-Ser-OH | 15 | GGGKDLEGKGGSAGSGSAGGSKYPYDVPDYAKS-[OH] |
| Pep1-Ser-NH2 | 16 | GGGKDLEGKGGSAGSGSAGGSKYPYDVPDYAKS-[NH2] |
| Pep2-RXXGXX | 17 | GGGRRLEGKEEDEKGSRASDRFRGLR-[OH] |
| Pep2-OH | 18 | GGGRRLEGKEEDEKGSRASDDFRDLR-[OH] |
| Pep2-NH2 | 19 | GGGRRLEGKEEDEKGSRASDDFRDLR-[NH2] |

Fluorescent proteins

| | | |
|---|---|---|
| sfGFP_Pep1- | 20 | MSMSKGEELFTGVVPILVELDGDVNGHKFSVRGEGEGDATN |
| CML | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSXGSGSXGGSKYPYDVPDYAKD-[OH], [X] = |
| | | N(6)-carboxymethyllysine |
| sfGFP_Pep1- | 21 | MSMSKGEELFTGVVPILVELDGDVNGHKFSVRGEGEGDATN |
| hCit | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKOKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSXGSGSXGGSKYPYDVPDYAKD-[OH], [X] = |
| | | homocitrulline |
| sfGFP_Pep1- | 22 | MSMSKGEELFTGVVPILVELDGDVNGHKFSVRGEGEGDATN |
| Ala | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSAGSGSAGGSKYPYDVPDYAKD-[OH] |
| sfGFP_Pep1- | 23 | MSMSKGEELFTGVVPILVELDGDVNGHKFSVRGEGEGDATN |
| Tyr | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSYGSGSYGGSKYPYDVPDYAKD-[OH] |
| sfGFP_Pep1- | 24 | MSMSKGEELFTGVVPILVELDGDVNGHKFSVRGEGEGDATN |
| NH2 | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKOKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSAGSGSAGGSKYPYDVPDYAKD-[NH2] |
| sfGFP_Pep1- | 25 | MSMSKGEELFTGVVPILVELDGDVNGHKFSVRGEGEGDATN |
| Ser-OH | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSAGSGSAGGSKYPYDVPDYAKS-[OH] |
| sfGFP_Pep1- | 26 | MSMSKGEELFTGVVPILVELDGDVNGHKFSVRGEGEGDATN |
| Ser-NH2 | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGK |
| | | DLEGKGGSAGSGSAGGSKYPYDVPDYAKS-[NH2] |
| sfGFP_Pep1- | 66 | MSMSKGEELFTGVVPILVELDGDVNGHKFSVRGEGEGDATNGKLTL |
| DOPA | | KFICTTGKLPVPWPTLVTTLTYGVQCFARYPDHMKQHDFFKSAMPE |
| | | GYVQERTISFKDDGTYKTRAEVKFEGDTLVNRIELKGIDFKEDGNILG |
| | | HKLEYNFNSHNVYITADKOKNGIKANFKIRHNVEDGSVQLADHYQ |
| | | QNTPIGDGPVLLPDNHYLSTQSKLSKDPNEKRDHMVLLEFVTAAGI |
| | | THGMDELYKSGGLPETGGGKDLEGKGGS[X]GSGS[X]GGSKYPYDV |
| | | PDYAKD-[OH], |
| | | [X] = 3,4-dihydroxyphenylalanine |
| sfGFP_Pep1- | 67 | MSMSKGEELFTGVVPILVELDGDVNGHKFSVRGEGEGDATNGKLTL |
| HNE | | KFICTTGKLPVPWPTLVTTLTYGVQCFARYPDHMKQHDFFKSAMPE |
| | | GYVQERTISFKDDGTYKTRAEVKFEGDTLVNRIELKGIDFKEDGNILG |
| | | HKLEYNFNSHNVYITADKQKNGIKANFKIRHNVEDGSVQLADHYQ |
| | | QNTPIGDGPVLLPDNHYLSTQSKLSKDPNEKRDHMVLLEFVTAAGI |
| | | THGMDELYKSGGLPETGGGKDLEGKGGS[X]GSGS[X]GGSKYPYDV |
| | | PDYAKD-[OH], |
| | | [X] = 4-hydroxy-2-nonenal |
| mTagBFP2- | 27 | MVSKGEELIKENMHMKLYMEGTVDNHHFKCTSEGEGKPYE |
| SRT-His6 | | GTQTMRIKVVEGGPLPFAFDILATSFLYGSKTFINHTQGIPDFF |
| | | KQSFPEGFTWERVTTYEDGGVLTATQDTSLODGCLIYNVKIR |
| | | GVNFTSNGPVMQKKTLGWEAFTETLYPADGGLEGRNDMA |
| | | LKLVGGSHLIANAKTTYRSKKPAKNLKMPGVYYVDYRLERIKE |
| | | ANNETYVEQHEVAVARYCDLPSKLGHKLNSGGLPETGGHHH |
| | | HHHV |
| sfGFP-SRT- | 28 | MSMSKGEELFTGVVPILVELDGDVNGHKFSVRGEGEGDATN |
| His6 | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGHH |
| | | HHHHV |
| mCherry- | 29 | MVSKGEEDNMAIIKEFMRFKVHMEGSVNGHEFEIEGEGEG |
| SRT-His6 | | RPYEGTQTAKLKVTKGGPLPFAWDILSPQFMYGSKAYVKHP |
| | | ADIPDYLKLSFPEGFKWERVMNFEDGGVVTVTQDSSLODGE |
| | | FIYKVKLRGTNFPSDGPVMQKKTMGWEASSERMYPEDGAL |
| | | KGEIKQRLKLKDGGHYDAEVKTTYKAKKPVQLPGAYNVNIKL |
| | | DITSHNEDYTIVEQYERAEGRHSTGGMDELYKSGGLPETGGH |
| | | HHHHHV |
| sfGFP-Pep2- | 30 | MSMSKGEELFTGVVPILVELDGDVNGHKFSVRGEGEGDATN |
| RXXGXX | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGR |
| | | RLEGKEEDEKGSRASDRFRGLR-[OH] |
| sfGFP-Pep2- | 31 | MSMSKGEELFTGVVPILVELDGDVNGHKFSVRGEGEGDATN |
| OH | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGR |
| | | RLEGKEEDEKGSRASDDFRDLR-[OH] |
| sfGFP-Pep2- | 32 | MSMSKGEELFTGVVPILVELDGDVNGHKFSVRGEGEGDATN |
| NH2 | | GKLTLKFICTTGKLPVPWPTLVTTLTYGVQCFARYPDHMKQH |
| | | DFFKSAMPEGYVQERTISFKDDGTYKTRAEVKFEGDTLVNRIE |
| | | LKGIDFKEDGNILGHKLEYNFNSHNVYITADKQKNGIKANFKI |
| | | RHNVEDGSVQLADHYQQNTPIGDGPVLLPDNHYLSTQSKLS |
| | | KDPNEKRDHMVLLEFVTAAGITHGMDELYKSGGLPETGGGR |
| | | RLEGKEEDEKGSRASDDFRDLR-[NH2] |
| mTagBFP2- | 33 | MVSKGEELIKENMHMKLYMEGTVDNHHFKCTSEGEGKPYE |
| Pep2- | | GTQTMRIKVVEGGPLPFAFDILATSFLYGSKTFINHTQGIPDFF |
| RXXGXX | | KQSFPEGFTWERVTTYEDGGVLTATQDTSLODGCLIYNVKIR |
| | | GVNFTSNGPVMQKKTLGWEAFTETLYPADGGLEGRNDMA |
| | | LKLVGGSHLIANAKTTYRSKKPAKNLKMPGVYYVDYRLERIKE |
| | | ANNETYVEQHEVAVARYCDLPSKLGHKLNSGGLPETGGGRR |
| | | LEGKEEDEKGSRASDRFRGLR-[OH] |
| mTagBFP2- | 34 | MVSKGEELIKENMHMKLYMEGTVDNHHFKCTSEGEGKPYE |
| Pep2-OH | | GTQTMRIKVVEGGPLPFAFDILATSFLYGSKTFINHTQGIPDFF |
| | | KQSFPEGFTWERVTTYEDGGVLTATQDTSLQDGCLIYNVKIR |
| | | GVNFTSNGPVMQKKTLGWEAFTETLYPADGGLEGRNDMA |
| | | LKLVGGSHLIANAKTTYRSKKPAKNLKMPGVYYVDYRLERIKE |
| | | ANNETYVEQHEVAVARYCDLPSKLGHKLNSGGLPETGGGRR |
| | | LEGKEEDEKGSRASDDFRDLR-[OH] |
| mTagBFP2- | 35 | MVSKGEELIKENMHMKLYMEGTVDNHHFKCTSEGEGKPYE |
| Pep2-NH2 | | GTQTMRIKVVEGGPLPFAFDILATSFLYGSKTFINHTQGIPDFF |
| | | KQSFPEGFTWERVTTYEDGGVLTATQDTSLODGCLIYNVKIR |
| | | GVNFTSNGPVMQKKTLGWEAFTETLYPADGGLEGRNDMA |
| | | LKLVGGSHLIANAKTTYRSKKPAKNLKMPGVYYVDYRLERIKE |
| | | ANNETYVEQHEVAVARYCDLPSKLGHKLNSGGLPETGGGRR |
| | | LEGKEEDEKGSRASDDFRDLR-[NH2] |
| mCherry- | 36 | MVSKGEEDNMAIIKEFMRFKVHMEGSVNGHEFEIEGEGEG |
| Pep2- | | RPYEGTQTAKLKVTKGGPLPFAWDILSPQFMYGSKAYVKHP |
| RXXGXX | | ADIPDYLKLSFPEGFKWERVMNFEDGGVVTVTQDSSLODGE |
| | | FIYKVKLRGTNFPSDGPVMQKKTMGWEASSERMYPEDGAL |
| | | KGEIKQRLKLKDGGHYDAEVKTTYKAKKPVQLPGAYNVNIKL |
| | | DITSHNEDYTIVEQYERAEGRHSTGGMDELYKSGGLPETGGG |
| | | RRLEGKEEDEKGSRASDRFRGLR-[OH] |
| mCherry- | 37 | MVSKGEEDNMAIIKEFMRFKVHMEGSVNGHEFEIEGE |
| Pep2-OH | | GEGRPYEGTQTAKLKVTKGGPLPFAWDILSPQFMYGSK |
| | | AYVKHPADIPDYLKLSFPEGFKWERVMNFEDGGVVTVT |
| | | QDSSLQDGEFIYKVKLRGTNFPSDGPVMQKKTMGWE |
| | | ASSERMYPEDGALKGEIKQRLKLKDGGHYDAEVKTTYK |
| | | AKKPVQLPGAYNVNIKLDITSHNEDYTIVEQYERAEGRH |
| | | STGGMDELYKSGGLPETGGGRRLEGKEEDEKGSRASDDF |
| | | RDLR-[OH] |
| mCherry- | 38 | MVSKGEEDNMAIIKEFMRFKVHMEGSVNGHEFEIEGE |
| Pep2-NH2 | | GEGRPYEGTQTAKLKVTKGGPLPFAWDILSPQFMYGSK |
| | | AYVKHPADIPDYLKLSFPEGFKWERVMNFEDGGVVTVT |
| | | QDSSLODGEFIYKVKLRGTNFPSDGPVMQKKTMGWE |
| | | ASSERMYPEDGALKGEIKQRLKLKDGGHYDAEVKTTYK |
| | | AKKPVQLPGAYNVNIKLDITSHNEDYTIVEQYERAEGRH |
| | | STGGMDELYKSGGLPETGGGRRLEGKEEDEKGSRASDDF |
| | | RDLR-[NH2] |

Fluorescence polarization anisotropy probes

| | | |
|---|---|---|
| FL-Pep2-NH2 | 60 | [Fluorescein]-KEEDEKGSRASDDFRDLR-NH2 |
| FL-lib-SVV- | 61 | [Fluorescein]-KKYRYDVPDYSVV-NH2 |
| NH2 | | |
| FL-CycD1- | 62 | [Fluorescein]-KAATPTDVRDVDI-NH2 |
| NH2 | | |

Conjugates

| | | |
|---|---|---|
| Conjugate XI | 63 | [BET Bromodomain inhibitor JQ-1]- |
| | | YDVPDYSVV-NH2 |
| Conjugate XII | 64 | [BET Bromodomain inhibitor JQ-1-PEG]- |
| | | YDVPDYSVV-NH2 |
| Conjugate | 65 | [BET Bromodomain inhibitor JQ-1-alkyl]- |
| XIII | | YDVPDYSVV-NH2 |

Amino sacids were loaded on chlorotrityl resin to access C-terminal carboxylic acids or Rink amide to access C-terminal amides. Automated peptide elongation was carried out on a Multisyntech Syro I parallel synthesizer according to the general peptide methods. Peptide was cleaved from the resin using TFA/DODT/H2O (95:2.5:2.5, v/v) for 1 h. The resin was removed by filtration and the filtrate concentrated under reduced pressure. The solution was triturated with Et2O and centrifuged to obtain crude peptide. The crude peptide was dissolved in H₂O/CH₃N (1:1, v/v)+0.1% (v/v) TFA and purified using preparative HPLC.

### Recombinant Production of Sortase-Tagged Fluorescent Proteins

Codon-optimized fluorescent protein cDNAs carrying tandem C-terminal Sortase-A and hexahistidine tags were synthesized (Integrated DNA Technologies) and cloned into the pET28 backbone by Gibson Assembly (NEBuilder HiFi DNA Assembly Master Mix, NEB). E coli BL21 (DE3) cells transformed with each expression vector were grown in terrific broth (LLG) to OD600=0.6 and recombinant proteins were expressed for 6 h at 37° C. in presence of 0.5 mM Isopropyl-B-D-thiogalactoside (IPTG). Proteins were extracted by sonication (Branson) in low-salt nickel buffer (150 mM NaCl, 20 mM Tris, 5% Glycerol, 25 mM imidazole, pH8) supplemented with Leupeptin (1 μg/ml), Pepstatin A (1 μg/ml) and PMSF (0.5 mM). Lysates were cleared by centrifugation (30 min, 40′000 g, 4° C.) and fluorescent proteins were purified on a HisTrap FF column (Cytiva) by elution with 250 mM imidazole.

### Conjugation of Synthetic Peptide to Fluorescent Proteins Using Sortase

Peptide was dissolved in sortase reaction buffer (50 mM Tris, 150 mM NaCl, pH 7.4 at 4° C.). pH was adjusted to 7-8 using 2 M NaOH. Peptide (final concentration 1 mM) was mixed with mCherry/GFP (final concentration 75 μM). Sortase was added (final concentration 2 μM) and incubated at 4° C. for 18 h. Unreacted mCherry/GFP and cleaved sortag were removed by Ni-NTA purification. The flow-through was collected and immediately buffer exchanged to ion exchange buffer (25 mM Tris, pH 8.5) using a desalting column (Cytiva). The sample was further purified by anion exchange using a MonoQ column (Cytiva) with a gradient of 0-25% high salt buffer (25 mM Tris, 1 M NaCl, pH 8.5) in 25 column volumes. Fractions containing the product were pooled, buffer exchanged to sortase reaction buffer (supplemented with 0.5 mM TCEP) and concentrated.

### Synthesis of C-Terminal Amide PROTACs

Fmoc-Valine was loaded onto RINK-amide resin using the general procedure. Peptide was elongated using the standard Fmoc-SPPS conditions. The N-terminal Fmoc-group was removed by treatment with 20 vol % piperidine. Either JQ1-acid of formula X,

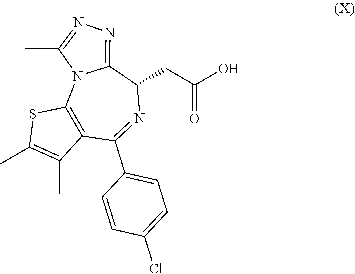 [SMILES: Cc1sc2c(c1C)C(c1ccc(Cl)cc1)=N[C@@H](CC(=O)O)c1nnc(C)n1-2]

PEG or alkyl-linker were coupled with HATU. For PEG and alkyl-linker containing peptides, Fmoc-group was removed by treatment with 20 vol % piperidine. JQ1-acid was coupled using HATU resulting in conjugates XI, XII and XIII. Said conjugates were cleaved from the resin and purified by preparative RP-HPLC.

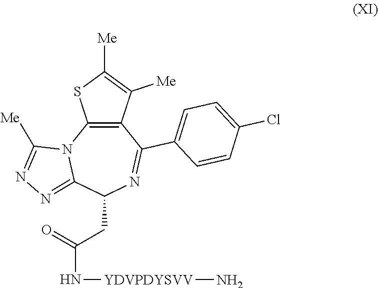

Conjugate XI (SEQ ID NO: 63) was obtained as a yellow-orange solid. HRMS (ESI): calculated for [C68H85ClN14O17S+H]+: m/z 1437.5705, found: m/z 1437.5696.

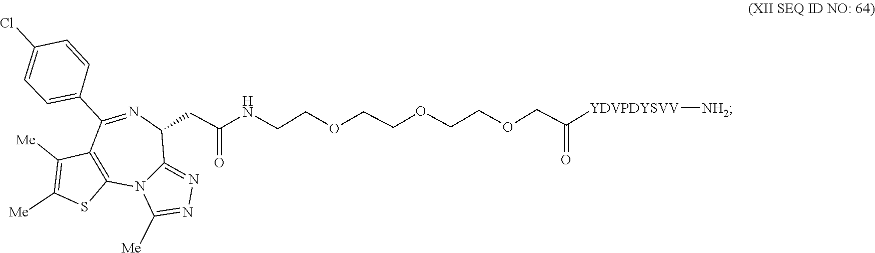

Conjugate XIII was obtained as a yellow-orange solid. HRMS (ESI): calculated for [C76H100ClN15O21S+H]+: m/z 1626.6706, found: m/z 1626.6746

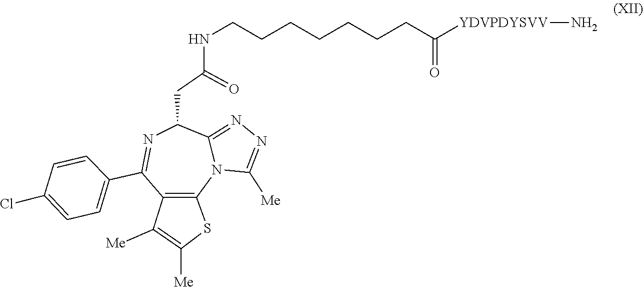

Conjugate XIII (SEQ ID NO: 65) was obtained as a yellow-orange solid. HRMS (ESI): calculated for [C76H100ClN15O18S+H]+: m/z 1578.6858, found: m/z 1578.6850

### Cell Culture and Fluorescent Reporter Stability Assays

HEK293T cells were cultured in DMEM containing Glutamax (Thermo Fisher Scientific) and K562 cells were cultured in RPMI 1640 containing Glutamax (Thermo Fisher Scientific) under standard conditions. Growth media were further supplemented with 10% (v/v) Fetal Bovine Serum (Thermo Fisher Scientific) and 100 U/ml Penicillin-Streptomycin (Thermo Fisher Scientific). Stably transduced cells were selected using 2 μg/ml Puromycin (Thermo Fisher Scientific), 500 μg/ml Geneticin (Thermo Fisher Scientific) or 10 μg/ml Blasticidin (Thermo Fisher Scientific) starting two days after transduction. Induction of doxycycline-dependent vectors was performed using 500 ng/ml doxycycline (Merck) every 48 hours. Cells were treated with epoxomicin (Merck), concanamycin A (Merck) or TAK243 (MedChem Express) as indicated.

For measuring stability of recombinant or semi-synthetic proteins, 1.5-2.0×10\hat{ }5 cells were nucleofected with 200 pmol of protein in 20 μl cuvettes using a 4D nucleofector device (Lonza) to according the manufacturer's recommendations. Following protein delivery, cells were left to recover at 37° C. for 30-45 minutes before measuring initial mean fluorescence intensity values (to) on a Attune N×T Flow Cytometer (Thermo Fisher Scientific). Subsequent measurements were performed at indicated time points. Baseline-fluorescence values of mock nucleofected cells receiving no protein were measured and subtracted for each time point and baseline-corrected fluorescence values were normalized to t0.

### Screen for Modified Amino Acid Degrons

The effect of each modification on protein degradation was measured in the human erythroleukemia cell line K562, which allows for efficient and uniform protein delivery via nucleofection. Flow cytometry measurements showed that sortase-tagged sfGFP alone was highly stable (t½>16 h), while introduction of a strong degron motif derived from the human ASCC3 C-terminus (—RXXGXX) led to rapid protein degradation.

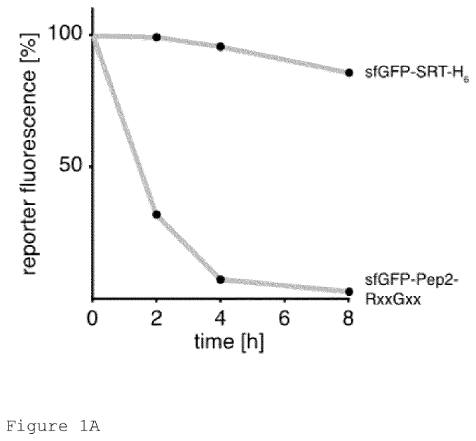

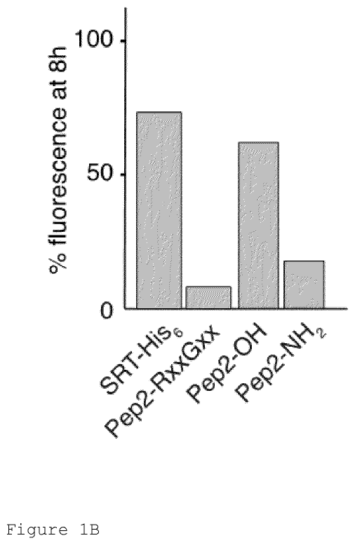

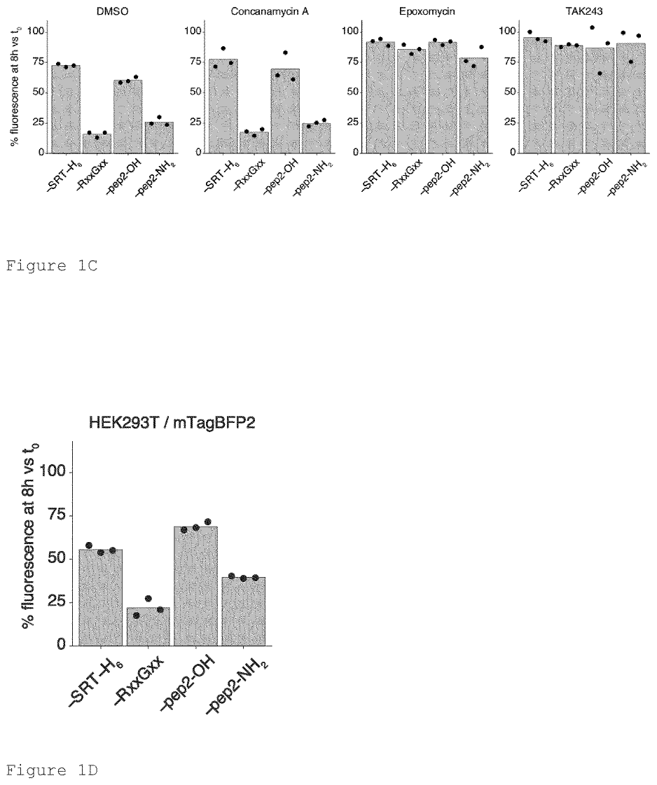

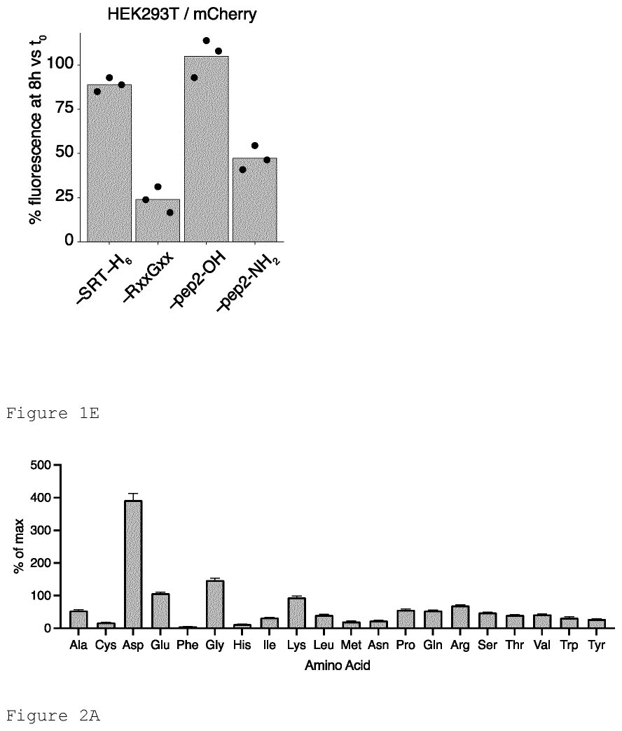

FIG. 1A shows a protein stability assay in K562 cells nucleofected with the unconjugated sfGFP reporter (sfGFP—SRT-His6; SEQ ID No. 28) or sfGFP carrying a C-terminal degron motif (sfGFP-Pep2-RXXGXX; SEQ ID No. 30). FIG. 1B shows the validation of terminal amidation as a degradation-inducing modification using an independent peptide context (Pep2) with (sfGFP-Pep2-NH₂, SEQ ID No. 32) or without terminal amide (sfGFP-Pep2-OH, SEQ ID No. 31) in a protein degradation time-course as in (b).

sfGFP carrying a primary amide on its C-terminus (sfGFP-Pepl-Ser-NH2; SEQ ID No. 26) was strongly destabilized in the context of the original screening peptide (FIG. 1B).

This effect could be fully blunted by inhibitors of total cellular ubiquitylation (TAK243) or the proteasome (epoxomicin), but by of lysosomal inhibition not acidification (concanamycin A) (FIG. 1C), suggesting that C-terminally amidated proteins are actively cleared from cells by the ubiquitin proteasome system. Lastly, conjugates of mTagBFP2 and mCherry carrying a primary C-terminal amide were also efficiently degraded when delivered to human embryonic kidney-derived HEK293T cells (FIG. 1D and FIG. 1E) establishing that C-terminal amidation induces protein degradation in different amino acid-, protein- and cell contexts.

### Gene Editing

FBXO31 knockout cells were generated by electroporation of cells with Cas9/sgRNA ribonucleoprotein particles as described previously (Lingeman, Jeans and Corn, 2017, Production of Purified CasRNPs for Efficacious Genome Editing.’, Current protocols in molecular biology, 120). In brief, in vitro transcription templates were generated by PCR using Q5 polymerase (New England Biolabs) and primers listed in supplementary table S1 and used for in vitro transcription by T7 RNA polymerase (NEB).

| | | | |
|---|---|---|---|
| | SEQ ID | | |
| Oligo_ID | NO.: | Sequence | note |

pRT-PCR

| | | | |
|---|---|---|---|
| qFBXO31-fwd | 39 | ACAGTGGAGTGCATGTACGG | quantification of FBXO31 |
| | | | expression |
| qFBXO31-rev | 40 | CACGTCCGAAACTCCTCCTG | quantification of FBXO31 |
| | | | expression |
| ACTB-fwd | 41 | GGGTCAGAAGGATTCCTATG | reference gene for qRT-PCR |
| ACTB-rev | 42 | GGTCTCAAACATGATCTGGG | reference gene for qRT-PCR |

genomic DNA amplification

| | | | |
|---|---|---|---|
| FBXO31.KO3-F1 | 43 | CTTTCCCTACACGACGCTCTTCCGATCT | amplification of target site for |
| | | TTCATCATCGGGTGGATGTAC | sgFBXO31.KO3 (SEQ ID NO: |
| | | | 53) with binding sites for NGS |
| | | | primers |
| FBXO31.KO3-R1 | 44 | GGAGTTCAGACGTGTGCTCTTCC | amplification of target site for |
| | | GATCTTCTGCAGCTGAGAATAGCAC | sgFBXO31.KO3 (SEQ ID NO: |
| | | | 53) with binding sites for NGS |
| | | | primers |
| TKOv3_PCR1_p | 45 | GAGGGCCTATTTCCCATGATTC | PCR1 of TKOv3 sgRNA library |
| LCKO2_fwd | | | from genomic DNA |
| TKOv3_PCR1_p | 46 | CAAACCCAGGGCTGCCTTGGAA | PCR1 of TKOv3 sgRNA library |
| LCKO2_rev | | | from genomic DNA |

deep sequencing library amplification

| | | | |
|---|---|---|---|
| TS_amplicon_ | 47 | AATGATACGGCGACCACCGAGATCT | generation of NGS libraries |
| NGS_fwd | | ACACGAGAGGTTACACTCTTTCCCT | from genomic DNA amplicons |
| | | ACACGACGCTCTTCCGATCT | (FBXO31.KO3-F1/R1) |
| TS_amplicon_ | 48 | CAAGCAGAAGACGGCATACGAGATG | generation of NGS libraries |
| NGS_rev | | GATCTGAGTGACTGGAGTTCAGACG | from genomic DNA amplicons |
| | | TGTGCTCTTCCGATC | (FBXO31.KO3-F1/R1) |
| TKOv3_PCR2_ | 49 | AATGATACGGCGACCACCGAGATCT | PCR2 of TKOv3 sgRNA library |
| 15(502)_fwd | | ACACGATATCGAACACTCTTTCCCT | for deep sequencing |
| | | ACACGACGCTCTTCCGATCTTTGTG | |
| | | GAAAGGACGAGGTACCG | |
| TKOv3_PCR2_ | 50 | AATGATACGGCGACCACCGAGATCT | PCR2 of TKOv3 sgRNA library |
| 15(503)_fwd | | ACACCGCAGACGACACTCTTTCCCT | for deep sequencing |
| | | ACACGACGCTCTTCCGATCTTTGTG | |
| | | GAAAGGACGAGGTACCG | |
| TKOv3_PCR2_ | 51 | CAAGCAGAAGACGGCATACGAGATC | PCR2 of TKOv3 sgRNA library |
| 17(701)_rev | | CGCGGTTGTGACTGGAGTTCAGACG | for deep sequencing |
| | | TGTGCTCTTCCGATCTACTTGCTAT | |
| | | TTCTAGCTCTAAAAC | |
| TKOv3_PCR2_ | 52 | CAAGCAGAAGACGGCATACGAGATA | PCR2 of TKOv3 sgRNA library |
| 17(704)_rev | | AGTCCAAGTGACTGGAGTTCAGACG | for deep sequencing |
| | | TGTGCTCTTCCGATCTACTTGCTAT | |
| | | TTCTAGCTCTAAAAC | |

Resulting RNA was purified using a spin-column kit (RNeasy mini kit, QIAGEN) and 120 μmol of sgRNA were complexed with 100 μmol of recombinant SpCas9 protein at RT for 20 minutes. Cas9 protein was obtained from the QB3 Macro Lab at UC Berkeley. Assembled sgRNA/Cas9 complexes were delivered to cells using a 4D nucleofector kit (Lonza) according to the manufacturer's instructions. Clonal cell lines were isolated by single-cell sorting using an SH-800 cell sorter (Sony) and characterized by genomic DNA extraction (Lucigen QuickExtract), PCR amplification of edited loci using Q5 polymerase (NEB) with genotyping and NGS primers listed above. Pooled next generation sequencing of edited loci was performed by the Genome Engineering and Measurement Lab of the Functional Genomics Center Zurich on a MiSeq sequencer (Illumina) with 150 bp paired-end reads. Deep sequencing reads were analyzed using CRISPResso2 (Clement, K. et al. (2019) ‘CRISPResso2 provides accurate and rapid genome editing sequence analysis’, Nature Biotechnology, 37 (3), pp. 224-226).

### Viral Transduction and Knockdown

Lentiviral vectors were packaged in HEK293T cells using standard methods (Stewart, S. A. et al. (2003) ‘Lentivirus-delivered stable gene silencing by RNAi in primary cells’, Rna, 9 (4), pp. 493-501). In brief, cells were incubated with plasmid DNA (transfer plasmid, pCMV-dR8.2 dvpr and pCMV-VSV-G at a weight-ratio of 4:2:1) and Polyethyleneimine (Mw ~25000 u) at a weight ratio 1:3 (total DNA to PEI). Viral supernatant was harvested at 48-72 hours post nucleofection by ultrafiltration and supplemented with 4 μg/ml polybrene. Gene knockdown was performed in a sub-clonal K562 cell line stably expressing dCas9-KRAB-BFP (see below). sgRNAs targeting near transcription start sites were chosen based on previously optimized design rules (Horlbeck, M. A. et al. (2016) ‘Compact and highly active next-generation libraries for CRISPR-mediated gene repression and activation.’, eLife, 5 (September 2016), pp. 1-20) and cloned into vector pCRISPRia.

| | | | |
|---|---|---|---|
| | SEQ | | |
| | ID | | |
| sgRNA.ID | NO.: | target Gene | protospacer |

CRISPR knockout

| | | | |
|---|---|---|---|
| sgFBXO31.KO3 | 53 | FBXO31 | ATCAGGTGGATCCTGAACAG |
| sgCD55.KO5 | 54 | CD55 | CTGGGCATTAGGTACATCTG |

CRISPRi (knockdown)

| | | | |
|---|---|---|---|
| sgCD59.Ci1 | 55 | CD59 | GCGCAGAAGCGGCTCGAGGC |
| sgFBXO31.Ci1 | 56 | FBXO31.1 | GTCTCCACCAGCAGCTCGGG |
| | | (isoform 1) | |
| sgFBXO31.Ci2 | 57 | FBXO31.1 | GgcTGGAGCGTGCGCACAGG |
| | | (isoform 1) | |
| sgFBXO31.Ci3 | 58 | FBXO31.1 | GCGGTGTGTGCTCGCCTTTG |
| | | (isoform 1) | |
| sgBFP.L2 | 59 | BFP (non- | GCTGAAGCACTGCACGCCAT |
| | | targeting | |
| | | control) | |

Puromycin was used to select for cells stably expressing sgRNAs. To validate knockdown efficiency, stably transduced sgRNA-expressing cells were harvested for RNA extraction (RNeasy Mini Kit, QIAGEN), reverse transcription (iScript Reverse Transcription Supermix, BioRad) and quantitative PCR (SsoFast EvaGreen Supermix, BioRad) analyzed on a QuantStudio 6 thermocycler (Thermo Fisher Scientific) using the ΔΔCT method.

### Generation of CRISPR- and CRISPRi-Competent Cell Lines

K562 cells competent for inducible gene knockout (iCas9) and genome-wide CRISPR screening were generated by transduction with vectors SRPB (pHR—SFFV-rtTA3-PGK-Bsr) and 3GCasT (pHR-TRE3G-hSpCas9-NLS-FLAG-2A-Thy1.1). Cas9-P2A-Thy1.1 expression was induced using doxycycline for 2 days and cells staining positive for Thy1.1 were isolated by single-cell sorting (Sony SH-800). Clonal lines were screened for cells that show no evidence of CD55 knockout following viral delivery of sgCD55.1 and efficient knockout following addition of doxycycline for 9 days, each by antibody staining and flow cytometry.

K562 cells were transduced with lentivirus encoding for dCas9-BFP—KRAB driven by an EF1α promoter (Addgene plasmid #102244). Six days post-transduction, single BFP⁺ cells were isolated by FACS. To assess the CRISPRi knockdown efficiency of each clone, cells were transduced with lentivirus encoding for sgRNA sgCD59.Ci1 (SEQ ID No. 55). 48 h later, cells were selected with puromycin. Seven days post-transduction, cells e stained with anti-CD59-APC and analyzed by flow were cytometry.

### CRISPR Screen and Next Generation Sequencing

iCas9 cells were transduced with the pooled lentiviral sgRNA library TKOv3 (Hart et al., 2017) at a multiplicity of infection (MOI) of about 0.3 as measured by serial dilution, puromycin selection and viability assay (CellTiter-Glo 2.0, Promega). Two pools of 1.2·10\hat{ }8 cells each were transduced, yielding ca 500-fold library coverage which was maintained throughout all cell culture steps. Cas9-expression was induced by addition of doxycycline for 5 days prior to delivery of reporter proteins to allow for efficient knockout while minimizing drop-out of essential genes (de Almeida et al., 2021). To isolate CTAP-clearance deficient cells ≥4 large-scale nucleofections were performed by combining 5·10\hat{ }7 cells, 2000 p mol C-terminally amidated sfGFP (sfGFP-Pep2-NH2 SEQ ID No. 32) and 2000 p mol unstable control protein mTagBFP2-PEP2-RXXGXX; (SEQ ID No. 33) in a 100 μl nucleofection reaction (4D nucleofector kit SE plus supplement SF1, Lonza). 14 hours following nucleofection, cells were transferred on ice and sorted into a CTAP deficient population (sfGFP high and mTagBFP2 low) and an unaffected control population (sfGFP low and mTagBFP low). Genomic DNA was extracted from snap-frozen sorted cells using the Gentra Pure kit (QIAGEN). sgRNA cassettes were isolated by two rounds of PCR using a previously published strategy with NEBNext Ultra II Q5 Master Mix (New England Biolabs) and primer pairs listed above.

Protospacers were quantified by deep sequencing using 21 initial dark cycles on an NextSeq 2000 device (Illumina) by the Genome Engineering and Measurement Lab of the Functional Genomics Center Zürich (GEML, FGCZ). sgRNA counts were retrieved using mageck count (MAGeCK v0.5.9.3) with default parameters (Li, W. et al. (2014) ‘MAGeCK enables robust identification of essential genes from genome-scale CRISPR/Cas9 knockout screens.’, Genome biology, 15 (12), p. 554). Enrichment of sgRNAs targeting the same gene in CTAP-deficient cells versus the control population was estimated using mageck test using a paired design for screening duplicates with option remove-zero both and otherwise default parameters.

### Protein Co-Immunoprecipitation (Co-IP)

For co-IP of semi-synthetic model substrates, cells were nucleofected 2 hours prior to harvest and treated with 2 μM MLN 4924 (MedChemExpress) and 500 nM epoxomicin (Merck) to block protein degradation and stabilize CRL complexes. 2 μM MLN4924 and 500 nM epoxomicin were added at all subsequent steps until cell lysis. Cells were harvested by centrifugation and washed in ice cold PBS followed by lysis in SCF—IP buffer (150 mM NaCl, 50 mM Tris-HCl, 20 mM NEM, 1 mM EDTA, 0.1% NP-40, 5% (v/v) glycerol, pH 7.5) supplemented with Halt protease inhibitor cocktail (Thermo Fisher Scientific) by vortexing and rotation for 30 min at 4° C. Debris was removed by centrifugation (4° C., 30 min, 20′000 g) and total protein concentrations measured using the Bradford protein assay (Thermo Fisher Scientific). Typically, 140 μg protein were incubated with 12 μl of anti-HA magnetic beads (Thermo Fisher Scientific) on a rotator wheel at 4° C. overnight. Beads were washed three times for 5 minutes in SCF—IP buffer and bound proteins were eluted twice using 0.1 M Glycine (pH 2), followed by addition of 0.2 volumes neutralization buffer (1.5 M NaCl, 0.5 M Tris-HCl, pH 8.0).

### Immunoblotting

Unless for co-IP studies, immunoblotting was performed on whole cell lysates in radioimmunoprecipitation buffer (RIPA, 0.5M Tris-HCl, 1.5M NaCl, 2.5% deoxycholic acid, 10% NP-40, 10 mM EDTA, pH 7.4) supplemented with protease inhibitors (Halt protease inhibitor cocktail, Thermo Fisher Scientific). Proteins were analyzed by polyacrylamide gel electrophoresis (PAGE) and wet transfer onto nitrocellulose membranes (0.2 μm pore size) using standard methods. Membranes were blocked with TBS-T (150 mM NaCl, 20 mM Tris, 0.1% (w/v) Tween-20, pH 7.4) containing 5% skimmed milk powder and incubated with primary antibodies diluted in TBST containing 5% bovine serum albumin (BSA) and 0.05% (w/v) sodium azide. Detection of primary antibodies was performed by standard methods using fluorescently labelled secondary antibodies and an Odyssey CLx scanner (LI-COR Biosciences).

### Recombinant Protein Production and Purification

Codon-optimized CDNAs of SKP1 and FBXO31 were generated by gene synthesis (Integrated DNA Technologies) and cloned into the bacterial expression vector pET28b. Expression was performed at 18° C. overnight in presence of 0.5 mM IPTG. Following bacterial cell lysis by sonication in buffer NBA (200 mM NaCl, 50 mM HEPES, 25 mM imidazole, 5 mM DTT freshly added, pH 8.0), recombinant protein was enriched on a HisTrap FF column (Cytiva) by elution with 250 mM imidazole. The histidine tag was removed by digestion with purified Ulp1 protease (pFGET19, Addgene plasmid #64697). Binary SKP1/FBXO31 complexes were further purified by anion exchange chromatography (HisTrap Q HP, Cytiva) in 50 mM HEPES (pH7.5) and 5 mM DTT on a linear gradient of 100-1000 mM NaCl followed by gel filtration (HiPrep Sephacryl S-100 HR, Cytiva) in 200 mM NaCl, 50 mM HEPES and 1 mM TCEP.

### Fluorescence Polarization Assays

Increasing concentrations of recombinant FBXO31/SKP1 complexes were mixed with 20 nM fluorescein-labeled peptides in FPA-buffer (150 mM NaCl, 25 mM HEPES, pH 7.5) and incubated for 30 minutes at room temperature. Fluorescence polarization was measured on a Victor Nivo Plate Reader (Perkin Elmer). Following subtraction of baseline signal, polarization levels were normalized to the highest signal of each pair of amidated and non-amidated peptides. Binding curves were fitted by least squares regression and half-maximal binding concentrations were extracted using Prism 9 (Graphpad) assuming one-site binding and no contribution from unspecific interaction. Competition binding assays were performed in the same way using a fixed ratio of 2 nM fluorescent probe and 200 nM FBXO31 and 0.67 nM to 100 μM competing ligand.

As mentioned above, fluorescein-modified peptides were prepared, in which the C-terminal amino acid was varied to any of the twenty proteinogenic amino acids. The binding affinity of FBXO31 to these amide-peptides as determined by fluorescence polarization measurement is shown in FIG. 2A. As shown, FBXO31 bound to all peptides with a marked preference for the ones varied with hydrophobic amino acids. With the Phe variation, the peptide bound with a K_{D} of 6 nM. Using charged amino acids for variation, the peptide bound with a markedly reduced affinity. Aspartic acid peptide being the weakest binder bound with an affinity of 370 nM.

### Pooled Peptide Library Synthesis and TIMS-TOF Analysis

Amino acids were mixed in the following molar ratio and dissolved in DMF to a total concentration of 500 mM to yield an isokinetic mixture for SPPS (Ostresh, J. M. et al. (1994) ‘Peptide libraries: Determination of relative reaction rates of protected amino in competitive couplings’, acids Biopolymers, 34 (12), pp. 1681-1689): Ala (3.4 mol %), Arg (6.5 mol %), Asn (5.3 mol %), Asp (3.5 mol %), Gln (5.3 mol %), Glu (3.6 mol %), Gly (2.9 mol %), His (3.5 mol %), Ile (17.4 mol %), Leu (4.9 mol %), Lys (6.2 mol %), Met (3.8 mol %), Phe (2.5 mol %), Pro (4.3 mol %), Ser (2.8 mol %), Thr (4.8 mol %), Trp (3.8 mol %), Tyr (4.1 mol %), Val (11.3 mol %). Amino acids were dissolved in DMF to a total concentration of 0.5 M and used as is for resin loading and peptide elongation steps.

Resin loading and peptide synthesis was performed as described in the general methods using the isokinetic amino acid mixture for variable positions. Automated peptide elongation was carried out on a Multisyntech Syro I parallel synthesizer according to the general peptide methods. The peptide was cleaved form the resin using TFA/DODT/H₂O (95:2.5:2.5, v/v) for 1 h. The resin was removed by filtration and the filtrate concentrated under reduced pressure. The solution was triturated with Et₂O and centrifuged to obtain crude peptide. Et₂O was repeated three times. The crude peptide was dissolved in H₂O/CH₃N (1:1, v/v) and lyophilized to obtain an off-white powder.

1 nmol of peptide library was incubated with a 5-fold excess of recombinant Strep II-tagged FBXO31/SKP1 complex in IP buffer 2 (200 mM NaCl, 25 mM HEPES pH 7.5, 5 mM DTT and pre-equilibrated magnetic Strep-Tactin XT beads (240 μl MagStrep type 3 XT beads per reaction, IBA life sciences). Reactions were incubated for 30 minutes at 4° C. on a rotator and washed 3 times with IP buffer 2. Peptide-Protein complexes were eluted with IP buffer 2 supplemented with 50 mM biotin (Merck). Eluted peptides and an equal amount of input library (1 pmol) were diluted 1:1 with triethylammonium bicarbonate (50 mM, pH 8.0). Peptides were isobarically-labeled with TMT 2-plex labels according to the manufacturer's instructions (Thermo Fisher Scientific). Input library was modified with label TMT-126 and eluted peptides with label TMT-127. Following labeling, peptides were combined 1:1. Combined peptides were desalted using 100 μL C18 ZipTips and dried (Savant SpeedVac). The dried peptides were resuspended in 5 vol % acetonitrile, 0.1 vol % formic acid and analyzed by LC-MS/MS analysis as described in the general methods using timsTOF-pro (Bruker).

### Test of Low Molecular Weight Probe

As discussed above, the C-terminally amidated tripeptide Ser-Val-Val-NH₂ (302.37 g/mol) was synthesized to test whether lower molecular weight probes could be derived based on the rules of C-terminus recognition by FBXO31, and it was found in competitive fluorescence polarization anisotropy measurements that this ligand can efficiently displace different fluorescent peptide probes from FBXO31, as shown in FIG. 2B (FL-lib-SVV—NH2: SEQ ID NO: 61; FL-CycD1-NH2: SEQ ID NO: 62; and FL-Pep2-NH₂: SEQ ID NO: 60).

1. A method of treatment of a human or an animal body comprising: treating the human or animal body with a compound A wherein the compound A comprises: a peptide binder specifically binding to the ubiquitin ligase adapter FBXO31 in the human or animal body, the compound A having a terminal binding region of the general formula (i)
U—NH₂ (i) with U being of the formula (ii) 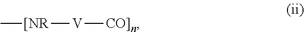
in which V is an organic moiety, and
R is hydrogen, a linear or branched, saturated or unsaturated C₁ to C₁₀ alkyl residue,
or forms together with a residue of V a ring system,
wherein U is a sequence of three amino acids and the C-terminal amino acid of U is selected from the group consisting of I, L, V, F, N, Y, S, T, A, W and M.

2. The method according to claim 1, wherein U is a sequence of canonical amino acids.

3. The method according to claim 1, wherein the C-terminal amino acid of U is selected from the group consisting of I, L, V, F, N, Y, S, T and A.

4. The method according to claim 1, wherein each of the three last C-terminal amino acids of U is selected from the group consisting of I, L, V, F, N and Y.

5. The method according to claim 1, wherein U contains a C-terminal motif of the amino acid sequence x₁x₂x₃, with
x₁ being selected from the group consisting of V, D, S and A;
x₂ being selected from the group consisting of I, V and T; and
x₃ being selected from the group consisting of I, L, V and F.

6. The method according to claim 5, wherein U contains a C-terminal motif of the sequence SVV.

7. The method according to claim 1, wherein compound A is of general formula (iii) 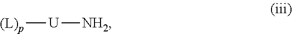
with L being a linker peptide comprising two or more natural amino acids and p being 0 or 1.

8. The method according to claim 7, wherein the linker peptide L comprises at least 3 amino acids.

9. The method according to claim 1, wherein compound A is of general formula (iv), 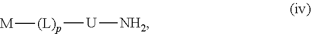
wherein M is a spacer moiety selected from the group consisting of linear or branched (C1-C₂₀)-alkylene, linear or branched (C₂-C₂₀)-alkenylene, linear or branched (C₂-C₂₀)-alkynylene, (C₃-C₂₀)-cycloalkylene, or any combination thereof, wherein one or more carbon atoms in said groups is optionally replaced with a heteroatom selected from O, S, N, NH or N(C₁-C₆)-alkyl.

10. The method according to claim 9, wherein the spacer moiety is a linear (C₁-C₁₀)-alkylene chain or PEG with 1 to 5 repeating units.

11. The method according to claim 1, wherein said use is the use as a therapeutic agent to induce selective intracellular degradation of a disease-associated protein.

12. The method according to claim 11, wherein compound A is used as a heterobifunctional degrader or a monovalent molecular glue of the disease-associated protein.

13. A conjugate or a multimer comprising
a) a compound A as defined in claim 1 and
b) a ligand moiety linked to compound A and containing a binding domain or structure capable of binding specifically to a disease-associated protein.

14. The conjugate or the multimer according to claim 13, wherein the disease-associated protein to be specifically bound by the binding domain or structure of the ligand moiety is selected from the group consisting of transcriptional regulators, oncogenic proteins and neurotoxic proteins.

15. The method according to claim 1, wherein the treatment includes a diagnostic method performed on the human or animal body or outside the human or animal body.


SEQUENCE LISTING (68 sequences)

SEQ ID NO: 1 - protein, 5 aa, synthetic construct
LPXTG

SEQ ID NO: 2 - protein, 6 aa, synthetic construct
LPETGG

SEQ ID NO: 3 - protein, 5 aa, synthetic construct
NPQTN

SEQ ID NO: 4 - protein, 5 aa, synthetic construct
NPKTG

SEQ ID NO: 5 - protein, 6 aa, synthetic construct
GSGSGS

SEQ ID NO: 6 - protein, 8 aa, synthetic construct
GSGSGSGS

SEQ ID NO: 7 - protein, 5 aa, synthetic construct
GGGGG

SEQ ID NO: 8 - protein, 33 aa, synthetic construct
  SITE 13: note = L-Dopa
  SITE 17: note = L-Dopa
GGGKDLEGKGGSXGSGSXGGSKYPYDVPDYAKD

SEQ ID NO: 9 - protein, 33 aa, synthetic construct; identical to SEQ ID NO: 8
  SITE 13: note = Hexanoyllysine
  SITE 17: note = Hexanoyllysine

SEQ ID NO: 10 - protein, 33 aa, synthetic construct; identical to SEQ ID NO: 8
  SITE 13: note = Carboxymethyllysine
  SITE 17: note = Carboxymethyllysine

SEQ ID NO: 11 - protein, 33 aa, synthetic construct; identical to SEQ ID NO: 8
  SITE 13: note = Homocitrulline
  SITE 17: note = Homocitrulline

SEQ ID NO: 12 - protein, 33 aa, synthetic construct
GGGKDLEGKGGSAGSGSAGGSKYPYDVPDYAKD

SEQ ID NO: 13 - protein, 33 aa, synthetic construct
GGGKDLEGKGGSYGSGSYGGSKYPYDVPDYAKD

SEQ ID NO: 14 - protein, 33 aa, synthetic construct; identical to SEQ ID NO: 12
  MOD_RES 33: note = amidation

SEQ ID NO: 15 - protein, 33 aa, synthetic construct
GGGKDLEGKGGSAGSGSAGGSKYPYDVPDYAKS

SEQ ID NO: 16 - protein, 33 aa, synthetic construct; identical to SEQ ID NO: 15
  MOD_RES 33: note = Amidation

SEQ ID NO: 17 - protein, 26 aa, synthetic construct
GGGRRLEGKEEDEKGSRASDRFRGLR

SEQ ID NO: 18 - protein, 26 aa, synthetic construct
GGGRRLEGKEEDEKGSRASDDFRDLR

SEQ ID NO: 19 - protein, 26 aa, synthetic construct
  MOD_RES 26: note = amidation
GGGRRLEGKEEDEKGSRASDDFRDLR

SEQ ID NO: 20 - protein, 280 aa, synthetic construct
  SITE 260: note = N(6)-carboxymethyllysine
  SITE 265: note = N(6)-carboxymethyllysine
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGKDLEGKGGSXGSGSXGGSKYPYDVPDYAKD

SEQ ID NO: 21 - protein, 280 aa, synthetic construct; identical to SEQ ID NO: 20
  SITE 260: note = homocitrulline
  SITE 265: note = homocitrulline

SEQ ID NO: 22 - protein, 280 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGKDLEGKGGSAGSGSAGGSKYPYDVPDYAKD

SEQ ID NO: 23 - protein, 280 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGKDLEGKGGSYGSGSYGGSKYPYDVPDYAKD

SEQ ID NO: 24 - protein, 280 aa, synthetic construct; identical to SEQ ID NO: 22
  MOD_RES 280: note = amidation

SEQ ID NO: 25 - protein, 280 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGKDLEGKGGSAGSGSAGGSKYPYDVPDYAKS

SEQ ID NO: 26 - protein, 280 aa, synthetic construct; identical to SEQ ID NO: 25
  MOD_RES 280: note = amidation

SEQ ID NO: 27 - protein, 253 aa, synthetic construct
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNSGGLPETGGHHHHHHV

SEQ ID NO: 28 - protein, 256 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGHHHHHHV

SEQ ID NO: 29 - protein, 252 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGLPETGGHHHHHHV

SEQ ID NO: 30 - protein, 273 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGRRLEGKEEDEKGSRASDRFRGLR

SEQ ID NO: 31 - protein, 273 aa, synthetic construct
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGGRRLEGKEEDEKGSRASDDFRDLR

SEQ ID NO: 32 - protein, 272 aa, synthetic construct
  MOD_RES 272: note = amidation
MSMSKGEELFTGVVPILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYKSGGLPETGGRRLEGKEEDEKGSRASDDFRDLR

SEQ ID NO: 33 - protein, 270 aa, synthetic construct
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNSGGLPETGGGRRLEGKEEDEKGSRASDRFRGLR

SEQ ID NO: 34 - protein, 270 aa, synthetic construct
MVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLNSGGLPETGGGRRLEGKEEDEKGSRASDDFRDLR

SEQ ID NO: 35 - protein, 270 aa, synthetic construct; identical to SEQ ID NO: 34
  MOD_RES 270: note = amidation

SEQ ID NO: 36 - protein, 269 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGLPETGGGRRLEGKEEDEKGSRASDRFRGLR

SEQ ID NO: 37 - protein, 269 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKSGGLPETGGGRRLEGKEEDEKGSRASDDFRDLR

SEQ ID NO: 38 - protein, 269 aa, synthetic construct; identical to SEQ ID NO: 37
  MOD_RES 269: note = amidation

SEQ ID NO: 39 - DNA, 20 bp, Homo sapiens
  source 1..20: mol_type = genomic DNA
acagtggagtgcatgtacgg

SEQ ID NO: 40 - DNA, 20 bp, Homo sapiens
  source 1..20: mol_type = genomic DNA
cacgtccgaaactcctcctg

SEQ ID NO: 41 - DNA, 20 bp, Homo sapiens
  source 1..20: mol_type = genomic DNA
gggtcagaaggattcctatg

SEQ ID NO: 42 - DNA, 20 bp, Homo sapiens
  source 1..20: mol_type = genomic DNA
ggtctcaaacatgatctggg

SEQ ID NO: 43 - DNA, 49 bp, Homo sapiens
  source 1..49: mol_type = genomic DNA
ctttccctacacgacgctcttccgatctttcatcatcgggtggatgtac

SEQ ID NO: 44 - DNA, 48 bp, Homo sapiens
  source 1..48: mol_type = genomic DNA
ggagttcagacgtgtgctcttccgatcttctgcagctgagaatagcac

SEQ ID NO: 45 - DNA, 22 bp, synthetic construct
  source 1..22: mol_type = other DNA
gagggcctatttcccatgattc

SEQ ID NO: 46 - DNA, 22 bp, synthetic construct
  source 1..22: mol_type = other DNA
caaacccagggctgccttggaa

SEQ ID NO: 47 - DNA, 70 bp, synthetic construct
  source 1..70: mol_type = other DNA
aatgatacggcgaccaccgagatctacacgagaggttacactctttccctacacgacgctcttccgatct

SEQ ID NO: 48 - DNA, 65 bp, synthetic construct
  source 1..65: mol_type = other DNA
caagcagaagacggcatacgagatggatctgagtgactggagttcagacgtgtgctcttccgatc

SEQ ID NO: 49 - DNA, 92 bp, synthetic construct
  source 1..92: mol_type = other DNA
aatgatacggcgaccaccgagatctacacgatatcgaacactctttccctacacgacgctcttccgatctttgtggaaaggacgaggtaccg

SEQ ID NO: 50 - DNA, 92 bp, synthetic construct
  source 1..92: mol_type = other DNA
aatgatacggcgaccaccgagatctacaccgcagacgacactctttccctacacgacgctcttccgatctttgtggaaaggacgaggtaccg

SEQ ID NO: 51 - DNA, 90 bp, synthetic construct
  source 1..90: mol_type = other DNA
caagcagaagacggcatacgagatccgcggttgtgactggagttcagacgtgtgctcttccgatctacttgctatttctagctctaaaac

SEQ ID NO: 52 - DNA, 90 bp, synthetic construct
  source 1..90: mol_type = other DNA
caagcagaagacggcatacgagataagtccaagtgactggagttcagacgtgtgctcttccgatctacttgctatttctagctctaaaac

SEQ ID NO: 53 - DNA, 20 bp, Homo sapiens
  source 1..20: mol_type = genomic DNA
atcaggtggatcctgaacag

SEQ ID NO: 54 - DNA, 20 bp, Homo sapiens
  source 1..20: mol_type = genomic DNA
ctgggcattaggtacatctg

SEQ ID NO: 55 - DNA, 20 bp, Homo sapiens
  source 1..20: mol_type = genomic DNA
gcgcagaagcggctcgaggc

SEQ ID NO: 56 - DNA, 20 bp, Homo sapiens
  source 1..20: mol_type = genomic DNA
gtctccaccagcagctcggg

SEQ ID NO: 57 - DNA, 20 bp, Homo sapiens
  source 1..20: mol_type = genomic DNA
ggctggagcgtgcgcacagg

SEQ ID NO: 58 - DNA, 20 bp, Homo sapiens
  source 1..20: mol_type = genomic DNA
gcggtgtgtgctcgcctttg

SEQ ID NO: 59 - DNA, 20 bp, Homo sapiens
  source 1..20: mol_type = genomic DNA
gctgaagcactgcacgccat

SEQ ID NO: 60 - protein, 18 aa, synthetic construct
  BINDING 1: note = Fluorescein
  MOD_RES 18: note = amidation
KEEDEKGSRASDDFRDLR

SEQ ID NO: 61 - protein, 13 aa, synthetic construct
  BINDING 1: note = Fluorescein
  MOD_RES 13: note = amidation
KKYRYDVPDYSVV

SEQ ID NO: 62 - protein, 13 aa, synthetic construct
  BINDING 1: note = Fluorescein
  MOD_RES 13: note = amidation
KAATPTDVRDVDI

SEQ ID NO: 63 - protein, 9 aa, synthetic construct
  BINDING 1: note = BET Bromodomain inhibitor JQ-1
YDVPDYSVV

SEQ ID NO: 64 - protein, 9 aa, synthetic construct
  BINDING 1: note = BET Bromodomain inhibitor JQ-1-PEG
  MOD_RES 9: note = amidation
YDVPDYSVV

SEQ ID NO: 65 - protein, 9 aa, synthetic construct
  BINDING 1: note = BET Bromodomain inhibitor JQ-1-alkyl
  MOD_RES 9: note = amidation
YDVPDYSVV

SEQ ID NO: 66 - protein, 280 aa, synthetic construct; identical to SEQ ID NO: 20
  SITE 260: note = 3,4-dihydroxyphenylalanine
  SITE 265: note = 3,4-dihydroxyphenylalanine

SEQ ID NO: 67 - protein, 280 aa, synthetic construct; identical to SEQ ID NO: 20
  SITE 260: note = 4-hydroxy-2-nonenal
  SITE 265: note = 4-hydroxy-2-nonenal

SEQ ID NO: 68 - protein, 13 aa, synthetic construct
  MOD_RES 13: note = amidation
  VARIANT 11: note = I L Y F H N V W P G A T Q R M K E D S
  VARIANT 12: note = I L Y F H N V W P G A T Q R M K E D S
  VARIANT 13: note = I L Y F H N V W P G A T Q R M K E D S
KKYRYDVPDYXXX